(19) United States
(12) Patent Application Publication
(10) Pub. No.: US 2025/0065026 A1
(43) Pub. Date: Feb. 27, 2025

(54) METHODS FOR DIRECT SODIUM REMOVAL IN PATIENTS HAVING HEART FAILURE AND RENAL DYSFUNCTION

(71) Applicant: Sequana Medical NV, Sint-Denijs Westrem (BE)

(72) Inventors: Jeffrey TESTANI, Guilford, CT (US); Ian CROSBIE, London (GB); Oliver GOEDJE, Strasslach (DE)

(21) Appl. No.: 18/946,828

(22) Filed: Nov. 13, 2024

Related U.S. Application Data
(63) Continuation-in-part of application No. 18/322,436, filed on May 23, 2023.
(60) Provisional application No. 63/487,863, filed on Mar. 1, 2023.
(60) Provisional application No. 63/685,232, filed on Aug. 20, 2024.

(51) Int. Cl. A61M 1/28; A61K 31/7004; A61K 31/716; A61K 33/00

(52) U.S. Cl. CPC A61M 1/287; A61K 31/7004; A61K 31/716; A61K 33/00

(57) ABSTRACT

Provided herein are infusates for direct sodium removal comprising sterile water, dextrose, icodextrin, and low to no sodium. Also provided herein are methods for conducing direct sodium removal therapy for a patient using the infusates provided herein.

### CROSS-REFERENCE TO RELATED APPLICATIONS

This application is a continuation-in-part of U.S. application Ser. No. 18/322,436 filed May 23, 2023, which claims benefit of U.S. Provisional Application No. 63/487,863, filed on Mar. 1, 2023, and claims benefit of U.S. Provisional Application No. 63/685,232, filed on Aug. 20, 2024, the entire contents of which are incorporated herein by reference.

### FIELD OF THE INVENTION

The present invention relates generally to methods of using infusates that are administered to a patient's peritoneal cavity to remove sodium and excess fluid from the body to alleviate fluid overload (“DSR therapy”). The above-incorporated patents describe such solutions for use in treating heart failure patients and patients having renal dysfunction. The inventions described herein are directed to specific protocols for conducting DSR therapy.

### BACKGROUND

U.S. Pat. No. 10,918,778 B2 describes DSR infusates and methods of use for removing excess fluid by the removal of sodium from heart failure patients having residual renal function. In particular, the DSR infusates are specially formulated to be instilled into a patient's peritoneal cavity to remove sodium and water through one or both of: 1) ultrafiltration and/or 2) diffusion of sodium ions down a steep concentration gradient between the infusate instilled into patient's peritoneal cavity and surrounding tissues and vessels. After sodium and ultrafiltrate accumulates in the peritoneal cavity, it may be drained in accordance with conventional peritoneal dialysis methods, or more preferably, transferred by an implantable pump to the patient's urinary bladder, from which it is subsequently voided.

The foregoing patent describes that excess fluid is eliminated from the body to maintain a relatively stable serum sodium concentration, by one or both of: 1) inducing fluid to migrate from the patient's body into the peritoneal cavity, from where it is eliminated and/or 2) enhancing the excretion of excess water via the kidneys through urination. Patients suffering from heart failure are prone to accumulate additional sodium in body tissues and suffer from increased fluid retention. Similarly, patients suffering from kidney failure also are prone to accumulate additional sodium in body tissues and suffer from increased fluid retention.

U.S. Pat. No. 5,589,197 to Shockley et al. describes a dialysate for use in peritoneal dialysis, wherein the sodium concentration is between about 35 to 125 meq/L. As discussed in that patent, such solutions may be used to transport sodium to the peritoneal cavity for subsequent removal. Problems encountered with early experience with such low sodium dialysates, included symptomatic drops in blood pressure, and dialysis disequilibrium syndrome, a potentially fatal complication resulting in cerebral edema, coma and death. See, e.g., Nakayma, Clinical Effect of Low Na Concentration Dialysate (120 mEq L) for CAPD Patients, PD Conference, San See, e.g., Zepeda-Orozco D, Quigley R., Dialysis disequilibrium syndrome, Pediatric Nephrology (Berlin, Germany) 2012; 27(12):2205-2211.

In accordance with existing standards of care, people with end-stage kidney disease generally undergo dialysis on a regular basis to remove metabolic toxins, and control sodium and fluid levels. The reported current average concentration of sodium in dialysate is generally about 132 mMol/L. See, e.g., Hecking M, Kainz A, Horl W H, Herkner H, Sunder-Plassmann G., Sodium setpoint and sodium gradient: influence on plasma sodium change and weight gain. American Journal of Nephrology 2011; 33(1):39-48; Mc Causland F R, Brunelli S M, Waikar S S., Dialysate sodium, serum sodium and mortality in maintenance hemodialysis. Nephrology Dialysis Transplantation 2012; 27(4):1613-8.

As reported in, e.g., Munoz Mendoza J, Sun S, Chertow G, Moran J, Doss S, Schiller B., Dialysate sodium and sodium gradient in maintenance hemodialysis: a neglected sodium restriction approach?, Nephrology Dialysis Transplantation 2011; 26(4):1281-7, experience with dialysate sodium concentrations higher than the patients' blood sodium levels show that such dialysates generally result in a net gain of sodium by the end of a dialysis session, which may result in increased fluid consumption and hypertension. Consequently, patients who undergo frequent hemodialysis sessions may experience chronic high sodium and fluid retention.

As described in the above-incorporated patents and applications, applicants have observed that eliminating fluid overload is a key clinical objective in managing heart failure and improving renal function in patients exhibiting renal dysfunction. Applicants have observed that achieving a stable reduction in fluid retention requires elimination of sodium from the body. Within certain ranges, patients having residual kidney function automatically will rebalance blood serum sodium concentration to maintain a constant serum osmolality and to maintain a stable sodium level, as described for example, in Guyton & Hall, Textbook of Medical Physiology. This is so because in patients with residual kidney function, the kidneys will remove excess water beyond that which might lead to hyponatremia.

It therefore would be desirable to develop methods of use in conducting DSR therapy for patients with fluid overload, such as patients having renal dysfunction or patients afflicted with heart failure. The desired DSR methods would assist such patients to achieve reductions in sodium and fluid overload not currently attainable with conventional dialysis techniques, while maintaining healthy serum sodium levels for such patients.

Accordingly, it would be desirable to provide methods of using DSR formulations, such as described in the above-incorporated patents and applications, and herein, to reduce fluid overload in patients suffering from renal dysfunction or heart failure.

### SUMMARY OF THE INVENTION

In accordance with the present invention, methods of using DSR infusates are provided for removing sodium and reducing fluid overload in patients having heart failure, renal dysfunction or both, while aiding maintenance of stable serum sodium levels. Based on pre-clinical and clinical testing, the results of which are reported in the above-incorporated patents and applications and herein, applicants have observed that instilling low or no sodium dextrose infusates, or small volume infusates containing dextrose and icodextrin, into the peritoneal cavities of patients can significantly reduce symptoms and co-morbidities associated with fluid overload. For example, in applicants' early testing, DSR formulations comprising a sterile solution of dextrose in a range of 10 to 15 weight percent, with no or low sodium concentration, resulted in removal of significant amounts of sodium (e.g., up to 5 gm) and fluid (e.g., up to 1.7 liter) during a two-hour dwell time. Similarly, small volumes of infusates containing 10 wt % dextrose and 30 wt % icodextrin provided comparable results. Applicants observed in those tests that patients' blood serum sodium levels remained substantially constant. To the extent that the amount of sodium migrating to the patient's peritoneal cavity causes a transitory serum sodium imbalance, the patients' kidneys caused additional water to be excreted to rebalance the blood serum sodium level. Applicants theorize that if sodium migrates to, and is removed from, the peritoneal cavity, serum sodium levels will be maintained by elimination of free water via the kidneys and/or release into the blood of sodium stored extravascularly, e.g., within the interstitium of skin and skeletal muscle or alternative storage locations within the body.

In accordance with one aspect of the present invention, applicants theorize that the amounts of sodium and water removed during a specified dwell period will be function of the concentration of sodium in the infusate, the osmolality and oncotic force of the infusate, and the ability of constituents of the infusate to migrate across through the peritoneum and into the patient's tissue and vascular system. For example, is it expected that an infusate with little or no sodium will create a steep gradient that causes sodium to migrate to the peritoneal cavity, whereas an infusate with a sodium concentration approaching isomolar with blood serum levels, e.g., 130 meq/ml, will create a much shallower gradient. Thus, sodium ions will move to the fluid in the peritoneal cavity asymptotically until that gradient disappears and the sodium concentration in the fluid in the peritoneal cavity becomes isotonic, e.g., 135-150 meq/l, provided the dwell period is sufficiently long.

Similarly, the osmolality and oncotic force of the infusate will determine its ability to drive water to the peritoneal cavity, and will vary based on the constituent in the infusate and its ability to migrate across the peritoneum. For example, it is known that dextrose provides high osmolality when a dextrose-containing infusate is instilled into the peritoneal cavity. However, because the dextrose molecules quickly migrate/disappear from the peritoneal cavity, the ability of the infusate to drive ultrafiltrate to the peritoneal cavity declines fairly rapidly. By comparison, it is known that the oncotic force of an icodextrin solution is maintained because the icodextrin molecules do not pass readily across the peritoneum, resulting in a sustained movement of ultrafiltrate. Accordingly, the ability of the infusate to drive excess fluid into the patient's peritoneal cavity also will determine the ability of the DSR infusate to move excess sodium and water to the peritoneal cavity for subsequent removal. In addition, the more water that migrates to the peritoneal cavity, the greater the amount of sodium that will migrate to the peritoneal cavity to render peritoneal fluid isotonic for sodium ions. Further still, it has been observed that a mixture containing a high concentration of icodextrin and lower concentration of dextrose may be instilled in a relatively small volume, such as 500 ml, and result in a many fold volume of water and sodium migration to the peritoneal cavity.

In accordance with one aspect of the present invention, methods of using specific DSR formulations are provided for use with renal failure patients and heart failure patients to reduce fluid overload, dispense with the need for or improve the performance of loop diuretics, and extract extravascularly-stored excess sodium. In one preferred method, a DSR infusate is employed that comprising an aqueous solution of 15-40 wt % icodextrin and up to 15 wt % dextrose. More preferably, the DSR infusate has a low sodium concentration, e.g., <120 meq/l or is sodium-free.

Applicants have observed in feasibility trials that a single dose of 500 ml of a DSR infusate instilled into a patient's abdomen via a previously implanted peritoneal dialysis catheter for a 24 hour dwell period, and then drained via the peritoneal catheter, may have a beneficial result. Initial testing in ten human renal failure patients demonstrated that the patients did not experience significant abdominal pain or hemodynamic irregularities, and that serum sodium levels remained stable. About 2.5 L of fluid isotonic for sodium, i.e., 135-154 meq/l, was removed from each patient (net of the DSR infusate infused), demonstrating a ratio of about 1:5 for instilled infusate as compared to net drained fluid at completion of the dwell period. It is expected that these patients may undergo one or more conventional hemodialysis or peritoneal dialysis sessions during the follow-up period to remove blood-borne toxins. DSR treatment in those patients can be repeated on an as-needed basis more frequently as well.

In accordance with a further aspect of the present invention, a method of using specific DSR formulations is provided for use with renal failure and heart failure patients to reduce fluid overload, eliminate or reduce the need for loop diuretics, and extract extravascularly-stored excess sodium. In accordance with this method, a DSR comprises an aqueous solution of 15-40 wt % icodextrin and 0-15 wt % dextrose, and more preferably, has a low sodium concentration, e.g., <120 meq/l or is sodium-free. In use, an amount of between 125 ml and 1.0 liter of the DSR infusate periodically is infused into a patient's abdomen via an implanted peritoneal dialysis catheter for a period of 14 to 28 days. During this period, the DSR infusate may be instilled once a week or up to once daily, and retained in the peritoneal cavity for a 4-24 hour dwell period. Upon completion of the dwell period, the infusate and ultrafiltrate are drained via the peritoneal catheter. Following the 14-28 day treatment period, the peritoneal catheter is removed and the patient is observed during a follow-up period, e.g., 3-month. If needed, additional DSR treatments may conducted during the follow-up period, and lowered doses of loop diuretics, if any, may be administered. The patients may be observed for a further 9-month period to determine whether further treatment with loop diuretics, or an increase in loop diuretics dose, is warranted to avoid fluid overload, and if so when.

Applicants expect that, based on the initial test results for human renal failure patients discussed above, fluid drained from a patient's abdomen at conclusion of each 4 to 24 hour dwell period should be isotonic for sodium, with a ratio of about 1:4 to 1:5 for instilled infusate as compared to net drained amounts. Further, preliminary testing suggests that the heart failure patients treated with the above-described protocol may not need loop diuretics, or may require much lower doses of loop diuretics, for an extended follow-up period after the DSR treatment. That preliminary testing further indicates that patients exhibit improvement to cardiovascular and renal parameters following DSR treatment.

Methods of instilling and removing the DSR infusates of the present invention also are provided. In particular, the DSR infusates may be instilled into, and together with the ultrafiltrate removed from, the peritoneal cavity using conventional implanted peritoneal dialysis tubing sets. Alternatively, an external pump may be used to instill infusate into the peritoneal cavity via a subcutaneous port having a catheter that extends in the peritoneal cavity; the infusate and sodium-laden ultrafiltrate then may be extracted via the catheter and port at the conclusion of the dwell period. As a further alternative, removal of infusate and sodium-laden ultrafiltrate may be accomplished with an implantable pump system such as described in commonly-assigned U.S. Pat. No. 9,956,336, the contents of which are incorporated herein by reference. That patent describes a system for ambulatory peritoneal dialysis in which an infusate infused into a patient's peritoneal cavity is removed after a predetermined time and/or at predetermined intervals via an implantable pump that transfers fluid accumulated in the peritoneum to the patient's bladder, where it may be subsequently voided.

In yet another aspect, provided herein is an infusate for direct sodium removal (DSR) therapy comprising: water, about 29% to about 31% icodextrin, about 9% to about 11% dextrose, and low to zero sodium.

In some embodiments, the infusate comprises about 9.5% to about 10.5% dextrose. In some embodiments, the infusate comprises 10% dextrose.

In some embodiments, the infusate comprises about 29.5% to about 30.5% icodextrin. In some embodiments, the infusate comprises 30% icodextrin.

In some embodiments, the infusate has a sodium concentration lower than 135 meq/l.

In some embodiments, the infusate further comprising a buffering agent.

In yet another aspect, provided herein is an infusate for direct sodium removal (DSR) therapy comprising: water, 30% icodextrin, 10% dextrose, and low to zero sodium a sodium concentration lower than 135 meq/l.

In some embodiments, the infusate further comprising a buffering agent.

In yet another aspect, provided herein is a method of conducting direct sodium removal (DSR) therapy for a patient, the method comprising: providing the infusate of any one of claim 1-8; during a 14-28-day treatment period, on a first treatment day, instilling between 125 ml and 1.0 liter of the infusate into a peritoneal cavity of the patient; retaining the infusate in the peritoneal cavity for a first 4-24-hour dwell period to induce sodium and ultrafiltrate to accumulate within the peritoneal cavity; and removing the infusate, sodium and ultrafiltrate from the peritoneal cavity.

In some embodiments, the method further comprises: during subsequent treatment days, up to once daily following the first treatment day, instilling between 125 ml and 1.0 liter of the infusate into the peritoneal cavity of the patient; retaining the infusate in the peritoneal cavity for subsequent 4-24-hour dwell periods during subsequent treatment days to induce sodium and ultrafiltrate to accumulate within the peritoneal cavity; and removing the infusate, sodium and ultrafiltrate from the peritoneal cavity.

In some embodiments, during the 14-28-day treatment period, on the first treatment day, instilling 500 milliliters of the infusate into the peritoneal cavity of the patient. In some embodiments, the method comprises, during subsequent treatment days, up to once daily following the first treatment day, instilling 500 milliliters of the infusate into the peritoneal cavity of the patient.

In some embodiments, the method comprises retaining the infusate in the peritoneal cavity for the first 24-hour dwell period to induce sodium and ultrafiltrate to accumulate within the peritoneal cavity. In some embodiments, the method comprises retaining the infusate in the peritoneal cavity for subsequent 24-hour dwell periods during subsequent treatment days to induce sodium and ultrafiltrate to accumulate within the peritoneal cavity.

In some embodiments, the method further comprises, prior to the first treatment day, reducing or suspending administration of loop diuretics to the patient as compared to the patient's loop diuretic regimen prior to the treatment.

In some embodiments, the method further comprises, following the 14-28-day treatment period removal, reducing or suspending administration of loop diuretics to the patient during a follow-up period of at least three months as compared to the patient's loop diuretic regimen prior to the treatment.

In some embodiments of the third aspect, the infusate is instilled into the peritoneal cavity using a peritoneal dialysis catheter. In some embodiments, removal of the infusate, sodium and ultrafiltrate from the peritoneal cavity following the first and subsequent 4-24 hour dwell period uses the peritoneal dialysis catheter. In some embodiments, removal of the infusate, sodium and ultrafiltrate from the peritoneal cavity following the first and subsequent dwell periods uses an implantable pump to transfer fluid from the peritoneal cavity to a bladder of the patient.

In some embodiments of the third aspect, the infusate is instilled into the peritoneal cavity via a subcutaneous port.

In some embodiments, removal of the infusate, sodium and ultrafiltrate from the peritoneal cavity following the first and/or subsequent 4-24-hour dwell periods uses an implantable pump to transfer fluid from the peritoneal cavity to a bladder of the patient.

In some embodiments, the dextrose rapidly crosses a peritoneal membrane of the patient and draws water into the peritoneal cavity to dilute the concentration of icodextrin, thereby reducing a rate at which the icodextrin is removed from the peritoneal cavity.

In yet another aspect, provided herein is a method of conducting direct sodium removal (DSR) therapy for a patient, the method comprising: providing the infusate described herein; during a 14-28 day treatment period, on a first treatment day, instilling between 500 milliliters of the infusate into a peritoneal cavity of the patient; retaining the infusate in the peritoneal cavity for a first 24-hour dwell period to induce sodium and ultrafiltrate to accumulate within the peritoneal cavity; removing the infusate, sodium and ultrafiltrate from the peritoneal cavity; during subsequent treatment days, up to once daily following the first treatment day, instilling between 500 milliliters of the infusate into the peritoneal cavity of the patient; retaining the infusate in the peritoneal cavity for subsequent 24-hour dwell periods during subsequent treatment days to induce sodium and ultrafiltrate to accumulate within the peritoneal cavity; and removing the infusate, sodium and ultrafiltrate from the peritoneal cavity.

### BRIEF DESCRIPTION OF THE DRAWINGS

Further features of the invention, its nature and various advantages will be more apparent from the accompanying drawings and the following detailed description of the preferred embodiments, in which:

FIG. 1 is an exemplary system for instilling DSR infusate into, and extracting infusate and ultrafiltrate from, a patient's peritoneal cavity using a subcutaneous port coupled to a catheter that extends into the patient's peritoneal cavity.

FIG. 2A-2B are, respectively, graphs showing the amounts of sodium removed in a first in-human (“FIH”) trial for 10 patients using a single dose of sodium-free 10 weight percent dextrose solution and a conventional peritoneal dialysis solution, sequentially administered via a conventional peritoneal dialysis catheter, as a function of dwell time, and sodium removal comparisons for each patient.

FIG. 3 is a table showing a durable effect post DSR-treatment for patients in a second study involving repeated DSR treatments, and who experienced a substantial reduction in need for diuretic drugs.

FIGS. 4A-4C are, respectively, graphs showing the evolution of observed changes in physiologic parameters for 7 patients during the second human trial.

FIG. 5 is a table showing durable reductions in loop diuretics resulting from a third human trial of DSR treatments in heart failure patients with severe fluid overload.

FIG. 6 is chart showing the sodium outputs diuretic challenges conducted at two week intervals after DSR treatments during the third human trial.

FIG. 7 is an exemplary protocol for treating a patient suffering from fluid overload from renal failure or heart failure.

FIGS. 8, 9 and 10 are graphs of sodium, icodextrin and glucose concentrations in the peritoneal fluid at the measurement time points during a fourth study conducted by instilling 500 ml of a no sodium infusate containing 10 wt % dextrose and 30 wt % icodextrin for 24 hours.

FIGS. 11, 12, 13 and 14 describe the pharmacokinetics of the icodextrin as a function of time during the dwell period for the fourth study.

FIG. 15 shows the ultrafiltration (UF) across various combinations of dextrose and icodextrin combinations in rats. The UF volume after 5 hours of dwell time with 10 ml infusate containing different concentrations and combinations of dextrose (D) and icodextrin (ico) is presented in the graph. Fluid was measured gravimetrically, and the instilled volume was assumed to be 10 g based on the 10 ml fluid injection. Bars and error bars represent mean and SEM, respectively. Asterisks indicate statistical significance at p<0.01. Std PD=standard peritoneal dialysis solution containing 7.5% icodextrin; H₂O=water.

FIGS. 16A-16D show the kinetics of UF (FIG. 16A), sodium removal (FIG. 16B), icodextrin metabolism (FIG. 16C) and total glucose (FIG. 16D) in sheep peritoneal dialysis fluid. FIG. 16C shows total glucose in ovine peritoneal fluid, while FIG. 16D shows total icodextrin in peritoneal fluid as a percentage of baseline icodextrin. Data points represent the mean value at each time point, error bars indicate SEM.

FIGS. 17A-17B show the kinetics of UF (FIG. 17A) and sodium removal (FIG. 17B) in pigs. Left panel shows ultrafiltration as determined by dilution of I-131 albumin added at infusion. Right panel shows that total sodium excretion across the peritoneal membrane was also higher with 30% icodextrin/10% dextrose in water peritoneal solution. Data points represent mean±SEM.

FIG. 18 shows the glucose concentration in porcine peritoneal fluid.

FIG. 19 shows a schematic of a Phase 1 open-label, single-arm, single-center, study of short-term safety, tolerability, and efficacy of a sodium-free 30% icodextrin/10% dextrose intraperitoneal (IP) solution in patients with end stage renal disease receiving chronic peritoneal dialysis (PD).

FIGS. 20A-20B shows measurements of perceived pain over the 24-hour dwell in humans. FIG. 20A shows the percentage of patients reporting pain from the start of infusion to 30 minutes after end of the intraperitoneal infusion. FIG. 20B shows the percentage of patients reporting pain from hour-1 to end of intraperitoneal fluid drain over the 24-hour dwell time. Blue=no pain; Red=mild pain (1 out of 4 on pain scale); Green=moderate pain (2 out of 4 on pain scale); Orange=severe pain (3 out of 4 on pain scale); Gray=extreme pain (4 out of 4 on pain scale).

FIGS. 21A-21B shows the kinetics of intraperitoneal and plasma icodextrin in humans who underwent a 24-hour dwell with a 30% icodextrin/10% dextrose IP solution. The IP (FIG. 21A) and plasma (FIG. 21B) icodextrin concentrations over 24 hours are shown as mean (SEM).

FIG. 22 shows the sodium and glucose concentration in peritoneal fluid over time in humans who underwent a 24-hour dwell with a 30% icodextrin/10% dextrose intraperitoneal IP solution. The IP fluid sodium (circles) and glucose (triangles) concentrations are graphed over 24 hours as mean (SEM) with the horizontal dotted line representing the serum sodium concentration over the same time period.

FIG. 23 shows the UF and sodium removal in humans who underwent a 24-hour dwell with a 30% icodextrin/10% dextrose IP solution. The net ultrafiltration volume (left panel), total sodium (mmol) in the ultrafiltrate (middle panel), and effective sodium concentration of the ultrafiltrate drained at the end of the 24-hour treatment period (right panel) are shown as median (IQR). Net ultrafiltration is calculated as the volume of IP drained at hour-24 after subtracting the 500 mL of 30% icodextrin/10% dextrose IP solution instilled. Effective sodium concentration in of the ultrafiltrate is calculated as total sodium in the ultrafiltrate (mmol) divided by the net ultrafiltration (mL).

### DETAILED DESCRIPTION OF THE INVENTION

The present invention is directed to infusate compositions and methods of treating fluid and/or sodium overload in patients with renal dysfunction, heart failure, or other disease associated with fluid overload. The infusate compositions and treatment methods are expected to be complimentary to conventional hemodialysis or peritoneal dialysis treatments and methods. Methods and compositions for treating heart failure patients suffering from fluid overload that have residual renal function are described in U.S. Pat. No. 10,918,778 B2, which is incorporated herein by reference in its entirety. The present invention is directed to specific methods for using DSR formulations to achieve significant, durable reduction in fluid overload and remove excess sodium. Based on preliminary data collected during first-in-man trials with exemplary DSR infusates, the methods and compositions of the present invention have been observed to improve sodium/fluid balance, reduce or even eliminate the need for loop diuretics, and improve parameters indicative of cardiovascular and renal health.

In accordance with one aspect of the present invention, a DSR infusate comprising an aqueous solution of up to 40 wt % icodextrin, and up to 15 wt % dextrose is instilled into a patient's peritoneal cavity using a peritoneal dialysis catheter, allowed to reside in the peritoneal cavity for a specified dwell period, and then drained via the peritoneal catheter.

In accordance with one aspect of the present disclosure, provided herein is an infusate for DSR that includes sterile water, about 29% to about 31% icodextrin, about 9% to about 11% dextrose, and low to zero sodium.

In some embodiments, the infusate includes about 9.5% to about 10.5% dextrose. In some embodiments, the infusate includes about 9.5%, about 9.6%, about 9.7%, about 9.8%, about 9.9%, about 10%, about 10.1%, about 10.2%, about 10.3%, about 10.4%, or about 10.5% dextrose. In some embodiments, the infusate includes about 9.5% dextrose. In some embodiments, the infusate includes about 9.9% dextrose. In some embodiments, the infusate includes about 10% dextrose. In some embodiments, the infusate includes about 10.1% dextrose. In some embodiments, the infusate includes about 10.5% dextrose.

In some embodiments, the infusate includes about 29.5% to about 30.5% icodextrin. In some embodiments, the infusate includes about 29.5%, about 29.6%, about 29.7%, about 29.8%, about 29.9%, about 30%, about 30.1%, about 30.2%, about 30.3%, about 30.4%, or about 30.5% icodextrin. In some embodiments, the infusate includes about 29.5% icodextrin. In some embodiments, the infusate includes about 29.9% icodextrin. In some embodiments, the infusate includes about 30% icodextrin. In some embodiments, the infusate includes about 30.1% icodextrin. In some embodiments, the infusate includes about 30.5% icodextrin.

In some embodiments, the infusate has a sodium concentration lower than 135 meq/l. In some embodiments, the infusate has a sodium concentration lower than 120 meq/l. In some embodiments, the infusate has a sodium concentration lower than 35 meq/l. In some embodiments, the infusate has zero or trace amounts of sodium.

In some embodiments, the infusate for DSR therapy includes water, 30% icodextrin, 10% dextrose, and low to zero sodium or a sodium concentration lower than 135 meq/l.

In some embodiments, the infusate includes one or more additional components. In some embodiments, the infusate further includes a high molecular weight biocompatible polymer solutions (average molecular weight Da>10,000). In some embodiments, the high molecular weight biocompatible polymer solution includes 0.5 g to 50 g of high molecular weight polymer per 100 ml of aqueous solution, and combinations thereof. In some embodiments, the infusate further includes electrolytes such as low amounts of magnesium or calcium salts, preservatives, ingredients having antimicrobial or antifungal properties, or buffering materials to control pH of the infusate.

In some embodiments, the infusate includes a buffering agent. Exemplary buffering agents that may be added to the infusate include, but are not limited to, borates, borate-polyol complexes, phosphate buffering agents, citrate buffering agents, acetate buffering agents, carbonate buffering agents, organic buffering agents, amino acid buffering agents, or combinations thereof.

In some embodiments, the infusate is formulated to be instilled into a peritoneal cavity of a patient using a peritoneal dialysis catheter, allowed to reside in the peritoneal cavity for a specified dwell period, and then drained via the peritoneal catheter.

In accordance with other embodiments of the disclosure, provided herein are methods for conducting DSR therapy for a patient using any of the DSR infusates described herein.

Specific methods have been investigated for treating fluid overload in renal failure and heart failure patients. In one exemplary preferred method, use of loop diuretics may be reduced or discontinued. On a first treatment day of a 14 to 28 day treatment period, a patient has between 125 ml and 1 liter of DSR infusate, as described above, instilled into the peritoneal cavity via an implanted peritoneal catheter. The infusate remains in the peritoneal cavity for a 4-24 hour dwell period, and then is drained. During a second treatment day, which may be anywhere from immediately after the 4-24 hour dwell period is completed, up to one day, two days, three days, four days, five days, or six days later, the patient again has between 125 ml and 1 liter of DSR infusate, as described above, instilled into the peritoneal cavity via an implanted peritoneal catheter. After a 4-24 hour dwell period, the infusate and accumulated fluid is drained from the patient's peritoneal cavity. Subsequent treatment days during the 14 to 28 day treatment period may occur up to daily, i.e., up to between 14 to 28 daily treatments and 3 or 4 weekly treatments, each employing a 4-24 hour dwell. A three-month follow-up preferably is conducted for the patient, during which loop diuretics may be reduced or discontinued until fluid accumulation is observed to occur. If reduced doses or no loop diuretics are needed during the initial follow-up period, the patient may be observed for an additional nine month period, during which DSR treatment may be repeated. During this follow-up period, loop diuretics are prescribed at reduced dosages, or not at all, until the patient is observed to again begin accumulating fluid. DSR treatment also may be repeated during the nine month follow-up period.

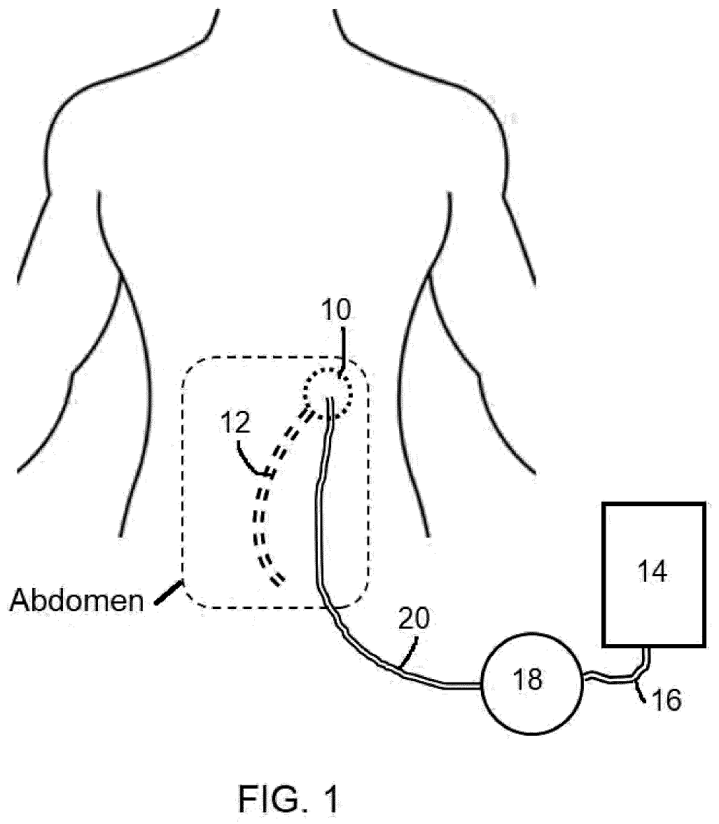

Referring to FIG. 1, an exemplary alternative method, DSR fluid may be instilled and later withdrawn from the peritoneal cavity (outlined in dotted line) using subcutaneous port 10 coupled to catheter 12 that extends into the peritoneal cavity. DSR infusate may be pumped from bag or reservoir 14 to external pump 18 via line 16 and then delivered to subcutaneous port 10 via external catheter 20. As will be understood by one of ordinary skill in the art of medical devices, subcutaneous port 10 may include a self-healing membrane to allow a sharpened needle port of catheter 20 to removably be inserted into subcutaneous port 10. Catheter 20 may be withdrawn from subcutaneous port 10 during the dwell period. At the conclusion of the dwell period, catheter 20 may again be coupled to subcutaneous port 10, and external pump 18 used to evacuate the DSR infusate and ultrafiltrate from the patient's peritoneal cavity. As a further alternative, fluid may be removed from the peritoneal cavity using an implanted pump that transfers the fluid to the patient's bladder, such as the alfapump commercialized by Sequana Medical NV of Ghent, Belgium.

As used in this disclosure, a no or low sodium DSR infusate has a sodium content generally less than 120 meq/L and includes infusates having zero or only trace amounts of sodium. Applicants theorize that a DSR solution having large proportion of icodextrin, e.g. 30-40 wt %, may perform satisfactorily with a significant weight percentage of sodium, e.g., up to a sodium concentration isotonic with blood plasma. Additional studies to confirm this hypothesis are planned.

In one exemplary embodiment, the DSR therapy is conducted with a DSR infusate comprising a no or low sodium solution consisting of sterile water containing 0 to 15 weight percent dextrose and up to 40 weight percent icodextrin. Such an exemplary DSR infusate composition is expected to remove both sodium and water. More generally, formulations suitable for DSR therapy may contain from zero to 50 grams of dextrose per 100 ml of aqueous solution, and more preferably, 5 to 10 weight percent dextrose; from 0.5 to 50 grams of icodextrin per 100 ml of aqueous solution, and more preferably 15 to 30 weight percent icodextrin. Suitable DSR infusates also may employ high molecular weight biocompatible polymer solutions (weight average molecular weight Da>10,000) having from 0.5 to 50 grams of high molecular weight polymer per 100 ml of aqueous solution, and combinations thereof. Additional constituents that may be substituted for the dextrose and/icodextrin include solutions containing between 0.5 and 50 grams of one or more of: xylitol, glycerol, hyperbranched polyglycerol, sorbitol, carnitine, taurine, amino acids, urea, polyacrylate, polyethyleneimine, and stevioside per 100 ml aqueous solution. The aqueous solution contains at least purified water, and in addition may include electrolytes such as low amounts of magnesium or calcium salts, preservatives, ingredients having antimicrobial or antifungal properties, or buffering materials to control pH of the infusate. icodextrin compositions generally may be preferable to dextrose compositions as icodextrin has been observed to experience a lower rate of uptake when employed in a peritoneal dialysis setting, and thus better preserves the peritoneal membrane compared to dextrose-based compositions.

The methods of using the DSR infusates optionally may include monitoring a patient's blood serum sodium level during a dwell period. For example, a blood sample may be taken during or after DSR therapy to monitor serum sodium level.

In some embodiments, the method for conducting DSR therapy for the patient includes: providing an infusate (e.g., an infusate described herein); during a treatment period, on a first day, instilling the infusate into a peritoneal cavity of the patient; retaining the infusate in the peritoneal cavity for a first dwell period to induce sodium and ultrafiltrate to accumulate within the peritoneal cavity; and removing the infusate, sodium and untrafiltrate from the peritoneal cavity. In some embodiments, the method further includes, during subsequent treatment days, up to once daily following the first treatment day, instilling the infusate into the peritoneal cavity of the patient; retaining the infusate in the peritoneal cavity for subsequent dwell periods during subsequent treatment days to induce sodium and ultrafiltrate to accumulate within the peritoneal cavity; and removing the infusate, sodium and ultrafiltrate from the peritoneal cavity.

In some embodiments, the treatment period is a 14- to 24-day treatment period. In some embodiments, the treatment period is a 14-day, 15-day, 16-day, 17-day, 18-day, 19-day, 20-day, 21-day, 22-day, 23-day or 24-day treatment period. In some embodiments, the treatment period is a 14-day treatment period. In some embodiments, the treatment period is an 18-day treatment period. In some embodiments, the treatment period is a 24-day treatment period. In some embodiments, the treatment period is longer than 24 days.

In some embodiments, the method includes instilling between 125 mL to 1.0 liter of the infusate into the peritoneal cavity. In some embodiments, the method includes instilling between 125 mL to 1.0 liter, between 200 mL to 900 mL, between 300 mL to 800 mL, between 400 mL to 700 mL, between 500 mL to 600 mL of the infusate into the peritoneal cavity. In some embodiments, the method includes instilling between 400 mL to 600 mL of the infusate into the peritoneal cavity. In some embodiments, the method includes instilling 125 mL, 150 mL, 200 mL, 250 mL, 300 mL, 350 mL, 400 mL, 450 mL, 500 mL, 550 mL, 600 mL, 650 mL, 700 mL, 750 mL, 800 mL, 850 mL, 900 mL, 950 mL or 1.0 liter of the infusate into the peritoneal cavity. In some embodiments, the method includes instilling 250 mL of the infusate into the peritoneal cavity. In some embodiments, the method includes instilling 500 mL of the infusate into the peritoneal cavity. In some embodiments, the method includes instilling 750 mL of the infusate into the peritoneal cavity.

In some embodiments the first and/or subsequent dwell periods are between 4 to 24 hours. In some embodiments, the first and/or subsequent dwell periods are 4-hour, 5-hour, 6-hour, 7-hour, 8-hour, 9-hour, 10-hour, 11-hour, 12-hour, 13-hour, 14-hour, 15-hour, 16-hour, 17-hour, 18-hour, 19-hour, 20-hour, 21-hour, 22-hour, 23-hour or 24-hour dwell periods. In some embodiments, the first and/or subsequent dwell periods are 12-hour dwell periods. In some embodiments, the first and/or subsequent dwell periods are 18-hour dwell periods. In some embodiments, the first and/or subsequent dwell periods are 24-hour dwell periods.

In some embodiments, the method for conducting DSR therapy for a patient includes: providing an infusate (e.g., an infusate described herein); during a 14-28 day treatment period, on a first treatment day, instilling between 500 milliliters of the infusate into a peritoneal cavity of the patient; retaining the infusate in the peritoneal cavity for a first 24-hour dwell period to induce sodium and ultrafiltrate to accumulate within the peritoneal cavity; removing the infusate, sodium and ultrafiltrate from the peritoneal cavity; during subsequent treatment days, up to once daily following the first treatment day, instilling between 500 milliliters of the infusate into the peritoneal cavity of the patient; retaining the infusate in the peritoneal cavity for subsequent 24-hour dwell periods during subsequent treatment days to induce sodium and ultrafiltrate to accumulate within the peritoneal cavity; and removing the infusate, sodium and ultrafiltrate from the peritoneal cavity.

In some embodiments, the infusate is instilled into the peritoneal cavity using a peritoneal dialysis catheter. In some embodiments, removal of the infusate, sodium and ultrafiltrate from the peritoneal cavity following the first and subsequent dwell period uses the peritoneal dialysis catheter.

In other embodiments, the infusate is instilled into the peritoneal cavity via a subcutaneous port.

In some embodiments, removal of the infusate, sodium and ultrafiltrate from the peritoneal cavity following the first and/or subsequent dwell periods uses an implantable pump to transfer fluid from the peritoneal cavity to a bladder of the patient.

In some embodiments, the method for conducting DSR therapy for a patient further includes, prior to the first treatment day, reducing or suspending administration of loop diuretics to the patient as compared to the patient's loop diuretic regimen prior to the treatment.

In some embodiments, the method for conducting DSR therapy for a patient further includes, following the treatment period, removal, reducing or suspending administration of loop diuretics to the patient during a follow-up period of at least three months as compared to the patient's loop diuretic regimen prior to the treatment.

In some embodiments, the dextrose rapidly crosses a peritoneal membrane of the patient and draws water into the peritoneal cavity to dilute the concentration of icodextrin, thereby reducing a rate at which the icodextrin is removed from the peritoneal cavity.

### EXAMPLES

### Example 1: Feasibility Studies

The assignee of the present invention sought and obtained permission from investigational review boards to conduct a first trial to assess the feasibility of removing sodium from patients using a single dose of sodium-free 10% dextrose DSR solution, and two further studies with heart failure patients to assess safety and tolerance to serial DSR treatments. The first trial was conducted with ten patients with end-stage renal disease who regularly underwent peritoneal dialysis with previously implanted peritoneal catheters. In the second study, which was completed with seven heart failure patients, a sodium-free 10% dextrose DSR solution repeatedly was instilled via a subcutaneous port and after a dwell time, removed via an implantable pump. In a third study, which was completed on ten heart failure patients with persistent congestion, a sodium-free 10% dextrose DSR solution repeatedly was instilled via a subcutaneous port and after a dwell time, removed via an implantable pump. In all of these trials, a solution of 10 weight percent dextrose in sterile water was used as the DSR infusate. Details and results of these trials are described with respect to FIGS. 1 through 5.

In the first study, patients receiving peritoneal dialysis for end-stage renal disease with functioning peritoneal dialysis catheters underwent randomization and crossover to open-label DSR solution (sodium-free 10% dextrose) or standard peritoneal dialysis solution (Dianeal Low-Calcium with 4.25% dextrose; Baxter; Deerfield, IL), each separated by 1 week. This phase 1 study was conducted in patients receiving prevalent peritoneal dialysis rather than normal subjects to avoid the risk that placing a peritoneal dialysis catheter would pose to a normal subject. Inclusion criteria were the following: actively undergoing peritoneal dialysis with a functioning peritoneal dialysis catheter implanted less than 3 years, patient age greater than 18 years and judged by the treating nephrologist to be at or above optimal volume status (i.e., not dehydrated). Exclusion criteria were uncontrolled diabetes mellitus with frequent episodes of severe hyperglycemia; systolic blood pressure <100 mm Hg; serum sodium <130 mEq/L; one or more episodes of peritonitis in the previous 6 months or active infection of the peritoneal dialysis catheter; anemia with hemoglobin <8 g/dL; serum bicarbonate <18 mEq/L; anuric renal failure; inability to give written informed consent or to follow the study protocol; and pregnant or lactating.

A 4.25% dextrose peritoneal dialysis solution was selected as the comparison solution because it is the most effective marketed peritoneal dialysis solution for fluid/sodium removal and has an osmolarity similar to that of 10% dextrose. Before instillation of the study fluid, there was a 30-minute drain of the abdomen with the patient assuming multiple positions during this time to ensure as complete drainage as possible. Given that this was a dialysis population with a tendency to develop acidosis, all patients were given 30 mEq sodium citrate/citric acid by mouth. Next, 1 L of either DSR infusate or standard peritoneal dialysis solution was infused into the peritoneum and left to dwell for 2 hours. The intraperitoneal volume was determined longitudinally using the indicator dilution technique with I-131-radiolabeled albumin (Daxor Inc), in addition to direct measurement of drained fluid at the end of the dwell. Vital signs, blood (every 30 minutes), and peritoneal fluid (every 15 minutes) were obtained serially throughout the protocol. Patients in the DSR group were given 50% of the ultrafiltration volume back at the end of the dwell in the form of intravenous normal saline to replace sodium/volume losses. The primary end point was safety/tolerability, defined as completion of the 2-hour dwell without significant discomfort or adverse events. The secondary efficacy end point was the difference in sodium removal between the DSR solution and standard peritoneal dialysis solution.

Ten patients completed the crossover study. The baseline characteristics for these patients are presented in the Table below.

| | |
|---|---|
| Characteristics | All Patients |

Demographics

| | |
|---|---|
| Age, y | 54 ± 12 |
| Male sex, n (%) | 70 (7) |
| White race, n (%) | 50 (5) |

Comorbidities, n (%)

| | |
|---|---|
| Diabetes mellitus | 30 (3) |
| Hypertension | 90 (9) |
| Heart failure | 10 (1) |

Physical examination

| | |
|---|---|
| Weight, lb, mean ± SD | 251 ± 72 |
| SBP, mm Hg | 144 (132-156) |
| Peritoneal Dialysis variables | |
| Peritoneal Dialysis Catheter vintage, years | 1.3 ± 0.9 |
| Automated Peritoneal Dialysis use, n (%) | 100 (10) |
| icodextrin use, n (%) | 30 (3) |
| Last fill, n (%) | 40 (4) |

Type of renal disease, n (%)

| | |
|---|---|
| Diabetic nephropathy | 2 (20) |
| Hypertensive nephrosclerosis | 1 (10) |
| Excessive NSAID use | |
| Systemic lupus erythematosus | |
| Nephrotic syndrome | |
| Immunoglobulin A nephropathy | |
| Granulomatosis with polyangitis-ESRD | |
| Polycystic kidney disease | |
| Failed allograft | |

Medications, n (%)

| | |
|---|---|
| Antihypertensives | 90 (9) |
| Loop diuretics | 60 (6) |
| Calcium channel blockers | 60 (6) |
| β-Blockers | 50 (5) |
| ACE inhibitors | 30 (3) |
| Thiazide-type diuretics | 20 (2) |
| Angiotensin II receptor blockers | 20 (2) |
| α-Agonists | 10 (1) |
| Insulin | 20 (2) |

Laboratory values, mean ± SD

| | |
|---|---|
| Sodium, mmol/L | 137.93.5 |
| Hemoglobin, g/dL | 10.21.3 |
| BUN, mmol/L | 5419 |
| Calcium, mmol/L | 1.11 ± 0.14 |
| Potassium, mmol/L | 4.2 ± 0.4 |

The primary end point, defined as completion of the 2-hour dwell without significant discomfort or adverse events, was met in all 10 patients. Overall, the treatment was well tolerated, with 2 of 10 patients reporting mild and short-duration cramping during instillation of the 10 wt % dextrose solution; one of those patients had similar cramping during the standard peritoneal dialysis solution instillation. There were no significant differences in blood pressure or peak heart rate between the 2 groups. Changes in plasma glucose were larger with the 10 wt % dextrose solution compared with standard peritoneal dialysis solution, with the most pronounced differences early in the dwell. However, differences in plasma glucose completely resolved after draining of the solution. No patients developed severe hyperglycemia at any time point in either group. The relative glucose absorption was the same between the 10 wt % dextrose and standard peritoneal dialysis solution; however, given the larger absolute amount of glucose in 10 wt % dextrose, the absolute quantity of glucose absorbed was larger with 10 wt % dextrose.

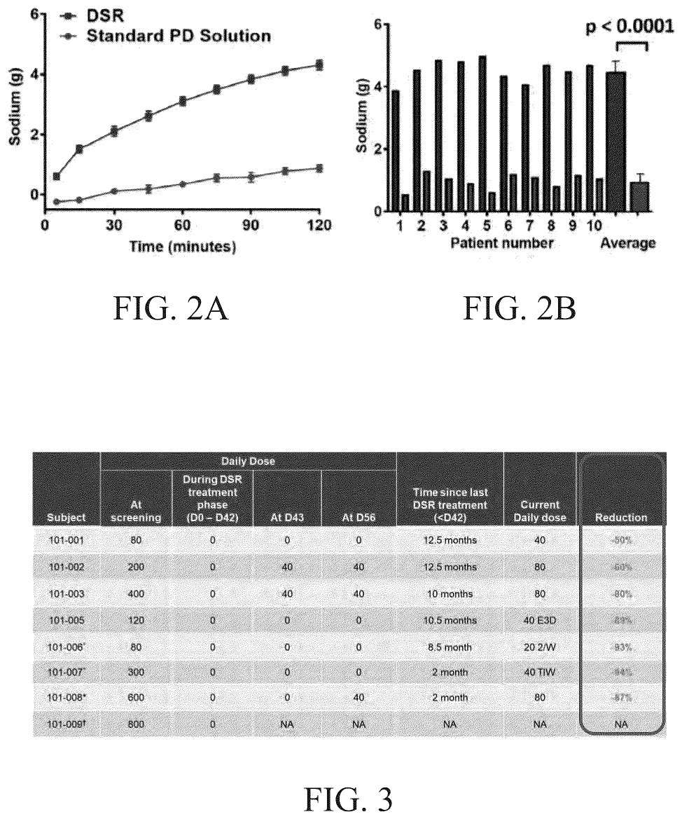

Serum sodium was not different between groups. Removal of off-target nonsodium electrolytes with DSR such as potassium (5.5±1.1 mmol), magnesium (1.7±2.5 mmol), phosphorus (1.9±0.6 mmol), and calcium (1.6±0.3 mmol) was negligible, and plasma electrolyte and chemistry parameters were stable throughout the dwell. As shown in FIG. 2A, the secondary efficacy outcome of superior sodium removal with sodium-free 10 wt % dextrose (4.5±0.4 g) compared with standard peritoneal dialysis solution (1.0±0.3 g) was met (P<0.001). In addition to a substantially higher average sodium clearance, the consistency of sodium removal was excellent. The absolute variability between individual patients' sodium removal and the average sodium removal was similar between DSR infusate and standard peritoneal dialysis solution, as depicted in FIG. 2B. However, because the total sodium removed in standard peritoneal dialysis solution was substantially lower, the relative variability between individuals was much higher with standard peritoneal dialysis solution, ranging from 8% to 75% of the average total sodium removal compared with 2% to 18% in patients receiving DSR infusate. Fluid removal was also greater with sodium-free 10 wt % dextrose, also with a high degree of consistency across patients.

The primary finding from the first human trial is that substantial sodium removal via the peritoneal membrane is feasible. In particular, it was observed that using 1 liter of sodium free 10 wt % dextrose solution, leveraged both diffusive and convective forces, and could remove >4 g sodium in 2 hours. The DSR therapy was well tolerated, with limited effect on plasma electrolyte levels, minimal off-target solute removal, and freedom from discomfort in the majority of human participants. The sodium removal was scalable, with substantially larger quantities of sodium removed by increasing the volume of 10 wt % dextrose cycled into the peritoneal space.

In the second study, the primary objective was to assess the safety and tolerability of serial treatment with DSR therapy in chronic stable diuretic resistant heart failure patients. A secondary objective of this study was to assess the impact of serial treatment with DSR therapy on parameters of cardiovascular and renal function, and diuretic response. In this study, DSR infusate was instilled into the patient's peritoneal cavities via subcutaneous port; seven patients completed the study. The implantable pump system developed by the assignee of the present application, Sequana Medical, N.V., was employed to move fluid from the patients' peritoneal cavities to their bladders. Patients were admitted into the second study, in accordance with the following criteria:

### Inclusion Criteria:

1. eGFR >30 ml/min/14.73 m2
2. Diagnosis of heart failure with one of the following:
   a. nt-proBNP >400 μg/ml (or BNP >100 μg/ml) and oral diuretic dose ≥80 mg furosemide equivalents OR
   b. Oral diuretic dose ≥120 mg furosemide equivalents
3. Stable diuretic dose for 30 days
4. Systolic blood pressure ≥100 mmHg
5. Determined by treating provider to be at optimal volume status

### Exclusion Criteria:

1. Serum sodium <135 mEq/L
2. Severe hyperkalemia or baseline plasma potassium >4.5 mEq/L
3. History of significant bladder dysfunction expected to interfere with ability of subject to tolerate DSR pumping into bladder
4. Uncontrolled diabetes with frequent hyperglycemia or Type 1 diabetes

The second study ran for a total of 6 weeks, plus a two-week follow-up. After screening, eight patients were implanted with the Alfapump® implantable pump about two weeks prior to initiation of the trial. Three days before the DSR trial was begun, each patient was given an intravenous administration of 40 mg of furosemide, followed by a 6-hour timed urine collection. At the outset of the DSR trial, all loop diuretics were stopped. During the following two weeks, the patients were resident in-hospital with diets controlled at 3 grams of sodium per day for the first week and 5 grams of sodium the second week. Patients then were permitted to return home. During the two week in-hospital stay and the following four weeks, the patients underwent DSR sessions up to three times a week using a sodium-free 10 weight percent dextrose DSR solution. At the conclusion of the six weeks of DSR sessions, the patients again underwent a diuretic challenge with intravenous administration of 40 mg of furosemide, followed by a 6-hour timed urine collection.

Baseline characteristics for the group of seven patients that completed the study are set forth in the table below:

| N = 7 | Result | Min:Max |
|---|---|---|
| Age-Years (Mean ± SD) | 62.0 ± 8.5 | 49:77 |
| Male-% | 100 | N/A |
| Height-cm (Mean ± SD) | 172.1 ± 5.4 | 163:182 |
| Weight-kg (Mean ± SD) | 75.6 ± 17.7 | 53.0:107.8 |
| BMI-kg/m² (Mean ± SD) | 25.4 ± 4.8 | 19.3:32.6 |
| Ejection Fraction-% (Mean ± SD) | 25.0 ± 2.5 | 20:28 |
| Nt-proBNP-pg/mL (Mean ± SD) | 3982.9 ± 2594.9 | 1536:7853 |
| Serum creatinine-μmol/L (Mean ± SD) | 106.9 ± 37.2 | 62.2:190.4 |
| Hematocrit-% (Mean ± SD) | 45.5 ± 5.9 | 36.6:55.2 |
| Furosemide equivalents-mg (Mean ± SD) | 254.3 ± 179.1 | 80:600 |

By the conclusion of the six-week study, all patients had remained off loop diuretics for the entire period. A neutral sodium balance was achieved (−1.3 grams) during the 2 week in-hospital period. Stable weight over the duration of the study also was achieved (75.6 to 75.5 kg). Most patients had down titration of DSR therapy to maintain constant weight; the volume of 10 wt % dextrose DSR solution was on average 750±348 ml/treatment.

As shown in table below, by completion of the study, study participants experienced a significant reduction in measured N-terminal-pro B-type natriuretic peptide (“NT-proBNP”) and creatinine levels, indicating improvement in cardio-renal function.

| Parameter | Pre-study | Post-study | Delta % |
|---|---|---|---|
| Nt-proBNP (pg/mL) | 3982.9 ± 2594.9 | 2780 ± 1544.1 | −30.2 |
| (Mean ± SD) | | | |
| Creatinine (μmol/L) | 106.09 ± 37.20 | 82.24 ± 17.27 | −23.1 |
| (Mean ± SD) | | | |
| Hematocrit-% (Mean ± SD) | 45.5 ± 5.9 | 43.40 ± 4.52 | −4.6 |
| Ejection Fraction-% | 25.0 ± 2.5 | 26.43 ± 3.99 | +5.7 |
| (Mean ± SD) | | | |
| Furosemide equivalents- | 254.3 ± 179.1 | 11.43 ± 19.52 | −95.5 |
| mg (Mean ± SD) | | | |
| Diuretic Challenge | 875.7 ± 281.1 | 1963.1 ± 464.6 | +124.2 |
| (40 mg furosemide)- | | | |
| 6H urine out (ml) | | | |
| Diuretic Challenge | 1.85 ± 1.02 | 5.37 ± 1.27 | +190.0 |
| (40 mg furosemide)- | | | |
| 6H Na out (gr) | | | |

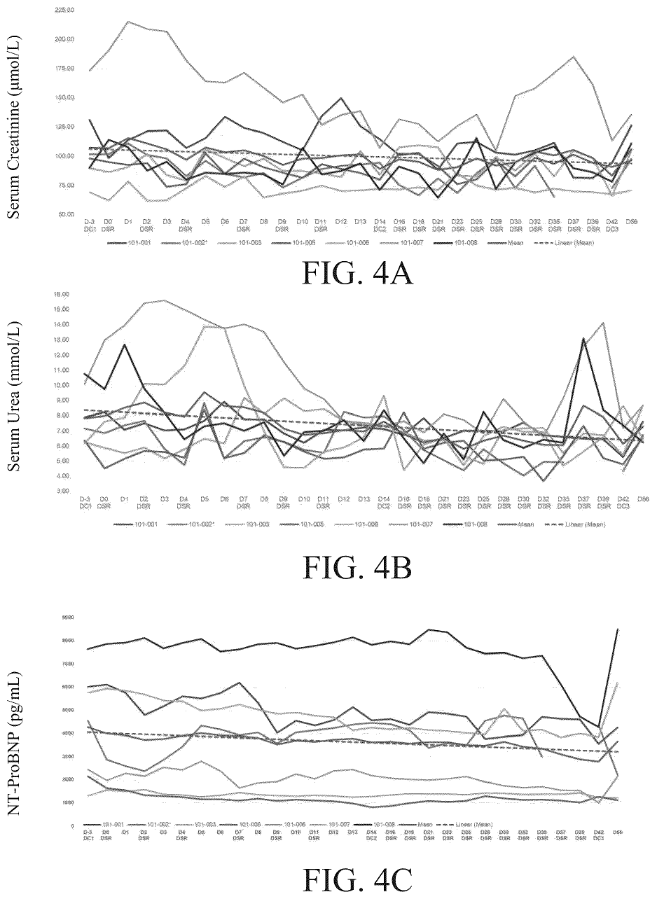

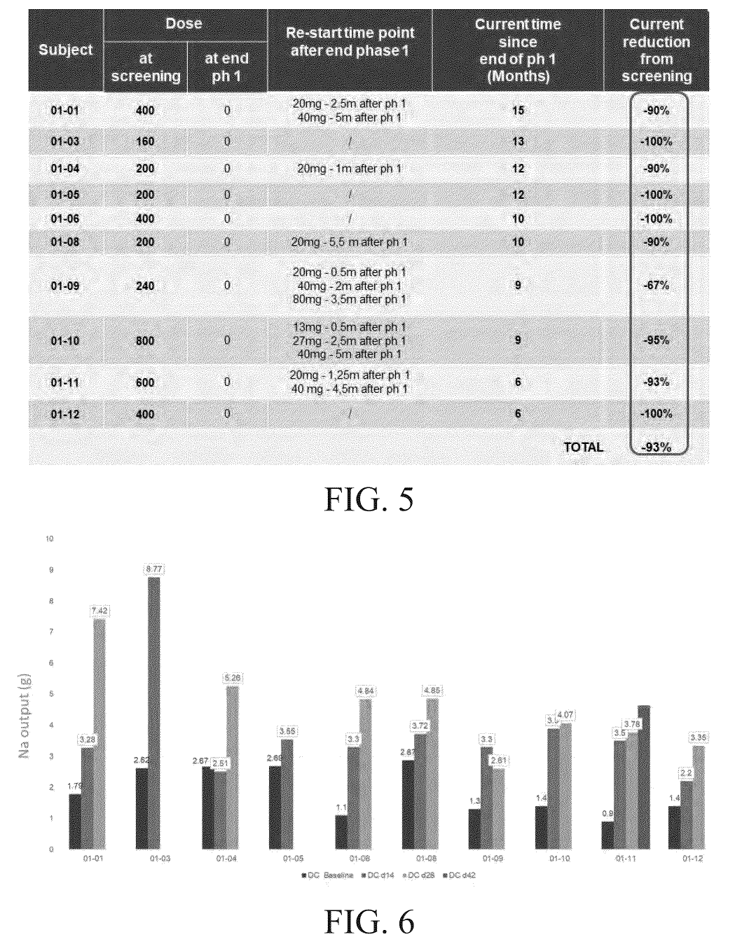

FIG. 3 is a table reporting the results of follow-up evaluations with seven of the study patients, and indicates that the effect of the DSR therapy in reducing diuretic resistance is durable, with patients experiencing a reduction between 94% and 50% of their daily diuretic dosing at 8 to 12 months following their last DSR session. FIGS. 4A, 4B and 4C depict the temporal trends of improvement for the study patients over the six-week period. This second human study demonstrated that six weeks of DSR therapy, using an implantable pump to remove the DSR solution and ultrafiltrate, was overall well tolerated and successfully maintained a neutral sodium balance and stable body weight, despite reduction of loop diuretics. The study also showed significant benefit to cardiovascular and renal function, as observed by meaningful improvement in NT-proBNP and renal function. In addition, diuretic resistance substantially and durably improved.

The third trial was conducted in which a sodium-free 10 wt % dextrose DSR infusate was used repeatedly in patients afflicted with heart failure and severe fluid overload. The primary objectives of this third study are to further demonstrate safety and tolerability of the Alfapump® DSR implant procedure and DSR therapy in subjects with residual congestion due to heart failure, while a secondary objective it so establish feasibility of the DSR infusate therapy to remove excess sodium and fluid to restore and maintain euvolemia without need for additional loop diuretic treatment.

The patient demographics for the third study are:

| Parameter | Result | Min:Max |
|---|---|---|
| Age-Years (Mean ± SD) | 64.4 ± 9.6 | 44:81 |
| Male | 10/10 (100%) | N/A |
| Weight-kg (Mean ± SD) | 82.9 ± 18.2 | 54.6:113.5 |
| Ejection Fraction-% (Mean ± SD) | 22.9 ± 3.8 | 18:28 |
| BMI-kg/cm² (Mean ± SD) | 28.3 ± 5.8 | 21:38 |
| Nt-proBNP-pg/mL (Mean ± SD) | 6628 ± 2483 | 2143:9000^{(*)} |
| Serum creatinine-μmol/L (Mean ± SD) | 141.7 ± 46.4 | 67.6:243.3 |
| eGFR-mL/min/1.73 m² (Mean ± SD) | 51.4 ± 23.4 | 23:110 |
| Furosemide equivalents-mg | 360.0 ± 196.8 | 160:800 |
| (Mean ± SD) | | |

An initial phase of the third study called for intensive DSR treatments, up to daily, for a two week period, with loop diuretics administered pro re nata (as needed). If the patient responded to the initial two week period, he would enter a second phase of the study, during which DSR treatments would be conducted once monthly in a four month follow-up period. If the patient did not sufficiently respond to the initial two-week course of DSR treatments, a further course of intensive DSR treatments would be conducted, before the patient moved to the second phase consisting of once monthly DSR treatment in a four month follow-up period.

The table below presents the results obtained at the conclusion of two weeks of intensive DSR treatments. As shown in the table, all patients were able to reduce their need for loop diuretics to zero.

| Parameter | Pre-Study | Post-Phase 1 | Delta % |
|---|---|---|---|
| Diuretic Challenge | 916.6 ± 219.6 | 1925.9 ± 512.9 | +110.1 |
| (40 mg furosemide)- | | | |
| 6H urine out (ml) (Mean ± SD) | | | |
| Diuretic Challenge | 1.9 ± 0.8 | 4.9 ± 1.9 | +163.4 |
| (40 mg furosemide)-6H Na | | | |
| out (gr) (Mean ± SD) | | | |
| Weight-kg (Mean ± SD) | 82.9 ± 18.2 | 76.3 ± 18.1 | −7.9 |
| Nt-proBNP (pg/mL) | 6628 ± 2483 | 4118 ± 2401 | −37.8 |
| (Mean ± SD) | | | |
| Creatinine (μmol/L) | 141.7 ± 46.4 | 129.0 ± 36.0 | −9.0 |
| (Mean ± SD) | | | |
| eGFR (mL/min/1.73 m²) | 51.4 ± 23.4 | 54.80 ± 16.9 | +6.6 |
| (Mean ± SD) | | | |
| Furosemide equivalents-mg | 360.0 ± 196.8 | 0.0 ± 0.0 | −100.0 |
| (Mean ± SD) | | | |

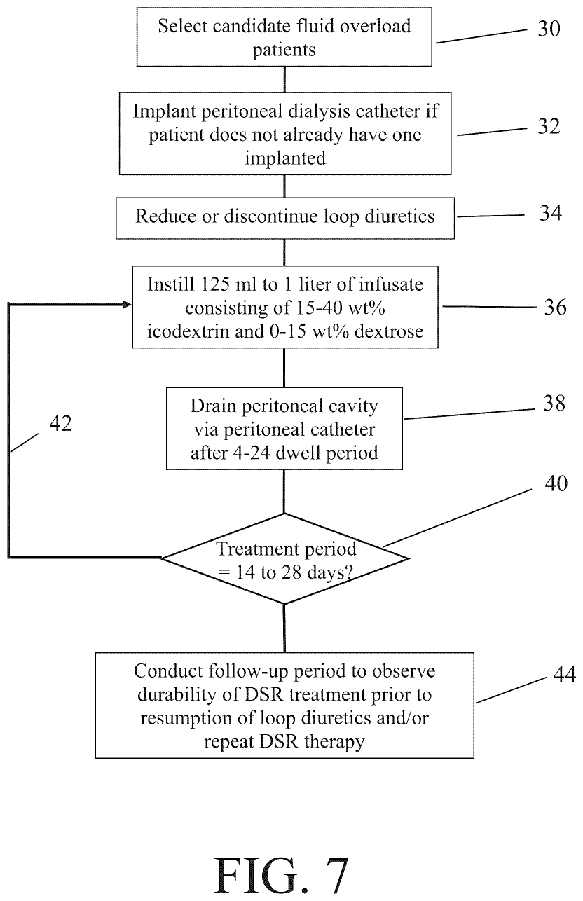

FIG. 5 is a table that presents the results of the follow-up period for the patients in the third trial. After several months hiatus after conclusion of the intensive DSR treatments, several patients have begun to take loop diuretics, but in much smaller doses that before they participated in the trial. For example, in the table of FIG. 5, patient 01-01 resumed a dose of 20 mg of furosemide two and half months after the conclusion of the first phase, which was increased to 40 mg after a further 2.5 months. After 15 months since conclusion of the two weeks of intensive DSR treatments, patient 01-01 continues to experience a 90% reduction in furosemide dosage compared to that at the outset of the study. Overall, as shown in FIG. 5, the group of patients experienced reductions of from 67% to 100% in use of loop diuretics, which reduction has been durable up to more than a year after the intensive treatment.

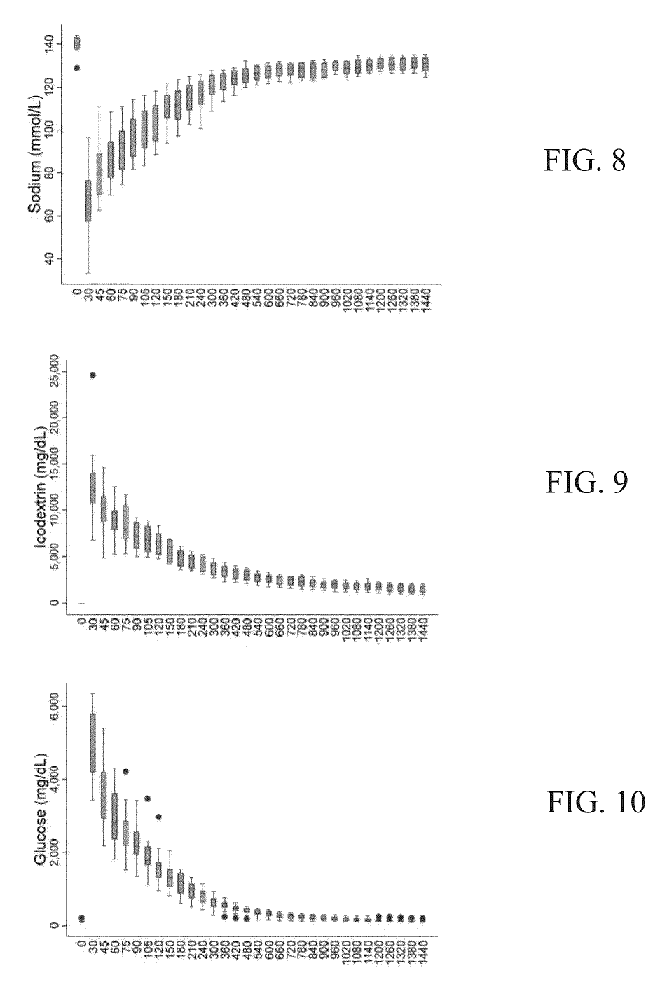

FIG. 6 is a chart showing sodium output data during 6-hour diuretic challenges conducted for the patients in the third trial, at baseline and two-week intervals after DSR treatment. Many patients repeated the challenge after four weeks, and one patient repeated the challenge at six weeks. During each diuretic challenge, each patient was administered a dose of 40 mg of furosemide, and sodium output in the patient urine was measured over the following six hours. As indicated in FIG. 6, the sodium output for almost of all patients after the DSR treatments more than doubled from the baseline sodium output.

Based on the results of three human trials discussed above, which employed a single composition of DSR infusate, i.e., sodium-free 10 wt % dextrose, it is theorized that DSR therapy interspersed with a renal failure patient's regularly conducted dialysis sessions may substantially enhance long-term efficacy of loop diuretics, permit the administration of reduced doses, and arrest and possibly reverse renal disease and heart failure. Similarly, for heart failure patients, episodes of DSR therapy can eliminate excess fluid, improve cardiovascular and renal performance and permit reduced or zero doses of loop diuretics for sustained periods following DSR therapy. In particular, DSR therapy may enable a patient's physician to gradually reduce fluid and sodium overload over a period of several weeks to months, and thereby establish an improved sodium and fluid metabolism for the patient that ameliorates either renal failure or heart failure.

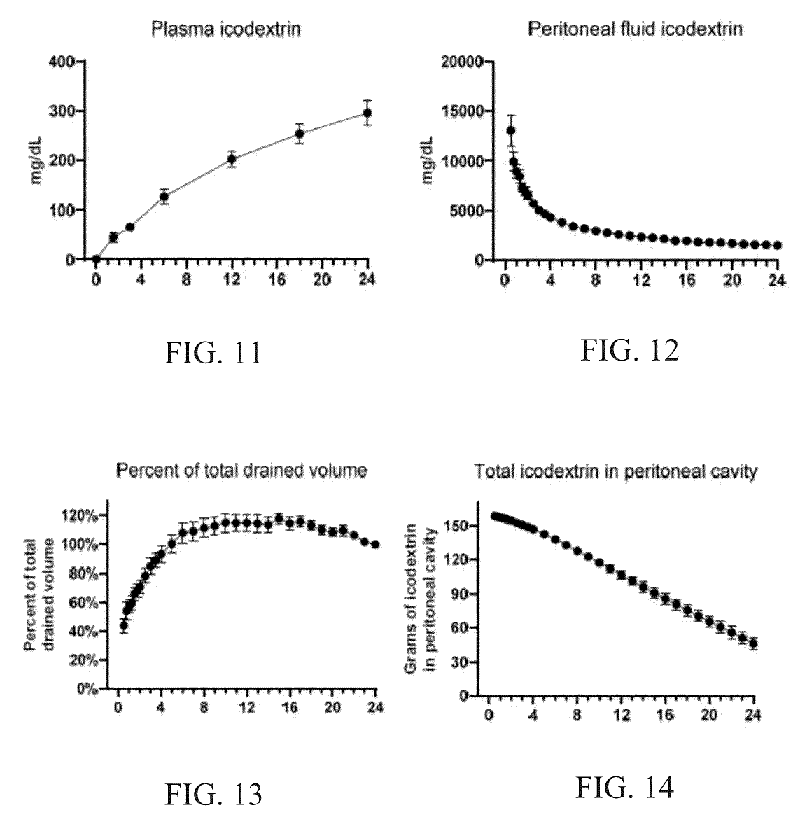

Turning now to FIG. 7, a method of using a DSR infusate comprising 15-40 wt % icodextrin and 0-15 wt % dextrose, and more preferably 30 wt % icodextrin and 10 wt % dextrose, to treat fluid overload patients, including renal heart failure patients, is described. The method begins at step 30, in which candidate fluid overload patients, such as renal or heart failure patients, are selected for DSR therapy. Inclusion and exclusion criteria for candidates may be as set forth above for the first in human trials. If the patient already has an implanted peritoneal dialysis catheter, that catheter may be used for DSR therapy; if not, a peritoneal dialysis catheter is implanted at step 32. At step 34, the patient's normal regimen of loop diuretics is reduced or suspended, so as not to confound the therapeutic benefits of the DSR therapy.

At step 36, on a first treatment day of a 14-to-28-day treatment period, between 125 ml and 1.0 liter of the DSR infusate is instilled into the patient's peritoneal cavity using the implanted peritoneal dialysis catheter. During a 4-to-24-hour dwell period, the patient may be monitored to confirm the patient does not experience undue discomfort and remains hemodynamically stable. Such monitoring may include verbal interactions with the patient, blood pressure, temperature and pulse rate measurements, and/or a blood sample to confirm serum sodium levels remain stable.

At step 38, after a 4-to-24-hour dwell period, the patient's peritoneal cavity is drained via the peritoneal dialysis catheter. As noted above, applicants' testing has demonstrated that net collected drainage volumes will be multiples of the amount of instilled infusate. The collected fluid also has been observed to be isotonic for sodium, and results in removal of several grams of sodium without significantly reducing blood serum sodium levels. In accordance with this method, at step 40, on a subsequent treatment day during the 14 to 28 day treatment period, which may immediately follow the first treatment day, or follow one, two, three, four, five or six days later, between 125 ml and 1.0 liter of DSR infusate again is instilled in the patient's peritoneal cavity, at steps 42, and steps 36-40 may be repeated. In this manner, the patient may undergo repeated DSR treatments during the 14-to-28-day treatment period. It is hypothesized that by undergoing such repeated processes, the patient's store of extravascular sodium may be depleted, thereby extending the durability of the beneficial effect of the DSR therapy.

At step 44, a follow-up period may be initiated to observe the long-term effect and durability of the DSR therapy. If during the follow-up period the patient begins to accumulate excess fluid, loop diuretics may again be prescribed for the patient at higher doses, and/or DSR treatment may be repeated. During applicants' clinical trials, it was observed that when loop diuretics were reinstituted, the dosages levels were significantly lower than before DSR therapy was begun.

### Example 2: Further Feasibility Studies

IP solutions for peritoneal dialysis (PD) in end-stage renal disease are primarily designed to enhance clearance of uremic toxins, which limits the amount of sodium and water that can be removed per unit of IP solution (EXTRANEAL (icodextrin) Peritoneal dialysis solution [package label]. Deerfield, IL: Baxter Healthcare Corporation; 2020; DIANEAL Peritoneal Dialysis Solution [package label]. Deerfield, IL: Baxter Healthcare Corporation; 2015). However, in heart failure (HF) and other edematous disorders, renal toxin clearance is adequate in most patients while renal sodium and water excretion are the primary aberration (Verbrugge F H, Guazzi M, Testani J M, Borlaug B A. Altered Hemodynamics and End-Organ Damage in Heart Failure: Impact on the Lung and Kidney. Circulation. Sep. 8, 2020; 142(10):998-1012. doi:10.1161/CIRCULATIONAHA.119.045409; Damman K, Testani J M. The kidney in heart failure: an update. European heart journal. Jun. 14, 2015; 36(23):1437-44. doi:10.1093/eurheartj/ehvOlO). As such, IP solutions can be designed with the principal goal of efficient salt and water clearance for application to these disease states (Morelle J, Sow A, Fustin C A, et al. Mechanisms of Crystalloid versus Colloid Osmosis across the Peritoneal Membrane. Journal of the American Society of Nephrology: JASN. July 2018; 29(7):1875-1886. doi:10.1681/ASN.2017080828; Fischbach M, Schmitt C P, Shroff R, Zaloszyc A, Warady B A. Increasing sodium removal on peritoneal dialysis: applying dialysis mechanics to the peritoneal dialysis prescription. Kidney international. April 2016; 89(4):761-6. doi:10.1016/j.kint.2015.12.032).

Previous studies provided a proof-of-concept that a sodium-free, 10% dextrose IP solution increased sodium removal four-fold compared to an equal volume of standard 4.25% dextrose commercially available PD solution (Rao V S, Turner J M, Griffin M, et al. First-in-Human Experience With Peritoneal Direct Sodium Removal Using a Zero-Sodium Solution: A New Candidate Therapy for Volume Overload. Circulation. Mar. 31, 2020; 141(13):1043-1053. doi:10.1161/CIRCULATIONAHA.119.043062). Building upon these promising observations, a dedicated IP solution that 1) was designed for enhanced sodium/water removal; 2) was concentrated in a small volume; and 3) produced an overall peritoneal safety superior to purely dextrose-based IP solutions was sought to be developed (Morelle J, Sow A, Fustin C A, et al. Mechanisms of Crystalloid versus Colloid Osmosis across the Peritoneal Membrane. Journal of the American Society of Nephrology: JASN. July 2018; 29(7):1875-1886. doi:10.1681/ASN.2017080828; Aanen M C, Venturoli D, Davies S J. A detailed analysis of sodium removal by peritoneal dialysis: comparison with predictions from the three-pore model of membrane function. Nephrol Dial Transplant. June 2005; 20(6):1192-200. doi:10.1093/ndt/gfh806). The goal was to create a biphasic solution that leveraged the safety and slow sustained ultrafiltration (UF) of icodextrin for its primary effect with the rapid UF of dextrose. With this approach, a low volume solution of highly concentrated icodextrin could rapidly “bloom” using dextrose-based UF into a larger volume of moderately concentrated icodextrin (prior to significant lymphatic uptake of the concentrated solution), thus providing slow continuous UF from the icodextrin component.

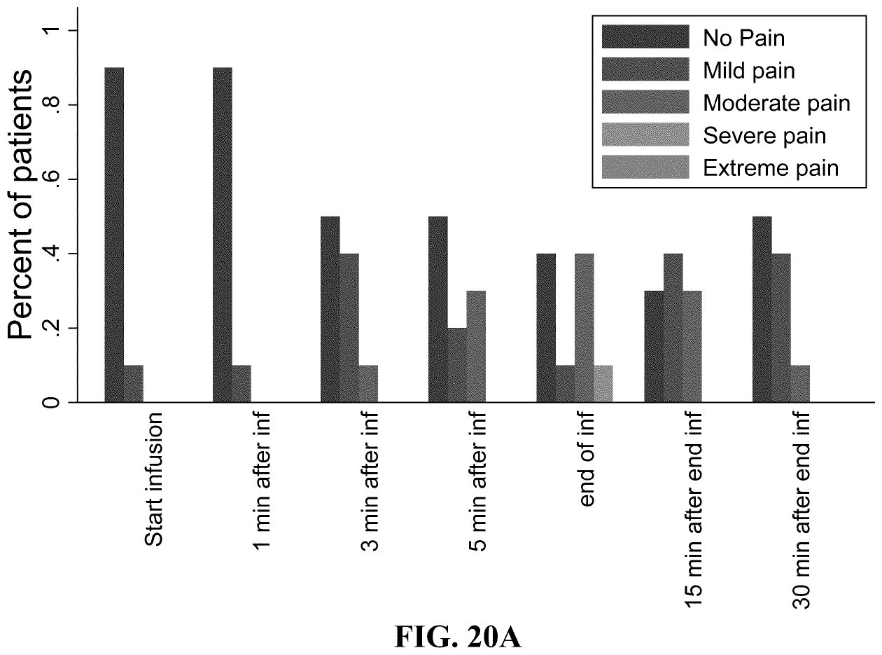

This example describes a further study was conducted using a DSR infusate consisting of sodium-free 30 wt % icodextrin solutions with up to 10 wt % dextrose, and the method described in FIG. 7. This study was designed to evaluate short-term safety and tolerability of sodium-free intraperitoneal 30% icodextrin and 10% dextrose solution in patients with chronic kidney failure undergoing peritoneal dialysis patients. The study was a an open-label, single-arm, single-center, study was conducted to evaluate the short-term safety, tolerability, and efficacy of a sodium-free 30% icodextrin/10% dextrose IP solution in 10 patients with end stage renal disease receiving chronic PD (FIG. 19; Chihuahua2022; NCT05780086). For the study, the 30% icodextrin and 10% dextrose IP solution (DSR Infusate 2.0) was manufactured by Infomed Fluids S.r.l. (Bucharest, Romania) in compliance with international standards for Chemistry, Manufacturing, and Controls product development. The interventional pilot study in humans was approved by the local IRB and registered at clinicaltrials.gov (NCT05780086). All patients provided written informed consent before any study procedures were performed.

During the study, ten patients had a 500 ml dose of the foregoing DSR infusate instilled into the peritoneal cavity via a PD catheter for a 24-hour dwell period. Upon completion of the dwell period, the peritoneal cavity was drained. The study was conducted as an open-label, single-center, exploratory study in patients with chronic kidney disease who previously had peritoneal dialysis catheters implanted.

### Inclusion Criteria:

1. Adults over the age of eighteen.
2. Patients with chronic kidney failure under peritoneal dialysis treatment and having a functional peritoneal dialysis catheter.
3. Patient's peritoneal dialysis prescription not changed in previous month (ongoing peritoneal dialysis as an outpatient or ongoing automated peritoneal dialysis).
4. Signed informed consent.
5. Peritoneal dialysis catheter and the transfer line compatible with the peritoneal dialysis bag (Luer male connector).
6. Patient clinically euvolemic as assessed by treating physician with no change to peritoneal dialysis prescription since performance of a peritoneal equilibrium test.
7. Stated wish to participate.
8. Patient's treatment covered by the Mexican Social Security Department.

### Exclusion Criteria:

1. Diabetes mellitus, type 1 or uncontrolled type 2 (HbAlc hemoglobin >8%).
2. Active infection or active bleeding.
3. Serum sodium prior to the study <130 mmol/l.
4. Serum bicarbonate prior to the study <18 mmol/l.
5. Peritoneal dialysis prescription using only solutions with 1.5% dextrose (lowest concentration available).
6. Hemoglobin <8 g/dl.
7. Peritoneal dialysis prescription using 4.25% dextrose solution at least once daily.
8. Membrane defect or mechanical defect.
9. Diastolic dysfunction with an increase in filling pressure according to the echocardiogram. (E/A ratio of >2.5 or an E/E ratio of >15).
10. Active or suspected peritonitis as assessed by treating physician.

Study Procedure: The peritoneal dialysis procedure began by connecting a dialysis bag containing the study solution (total volume of 500 ml DSR infusate) to the subject's peritoneal dialysis catheter. The catheter was disconnected and sealed once the total volume was infused. The subjects remained hospitalized during the entire 24-hour dwell time. Peritoneal liquid was sampled every 15 minutes during the first two hours, every 30 minutes during hour 3 and 4 and every hour until end of dwell time. Vital signs (blood pressure heart rate, respiration rate SpO2 and body temperature) were recorded every 30 minutes during the first 4 hours followed by measurements every hour until hour 24. Blood samples for point-of-care analysis were drawn at 3, 6, 12, 18, and 24 hours. Patients were monitored for any signs of adverse events during the entire treatment time.

Patients were given a standard diet containing 3 g of sodium and could consume water and savory snacks as desired to reduce the risk of hypovolemia. They were encouraged to increase liquid intake if point-of-care blood analyses revealed any signs of hypovolemia or given an intravenous Hartmann's solution (Lactated Ringer's Solution). Urine output over the 24 h dwell time was summed-up.

Safety was assessed by counting serious adverse events related to the infusate, monitoring of vital signs (blood pressure, heart rate, respiration rate, oxygen saturation, body temperature) and blood analyses (e.g., urea, creatinine, glucose, sodium, potassium, magnesium, calcium, phosphorus, bicarbonate and albumin) during dwell time (baseline, 3, 6, 12, 18, and 24 hours). Blood samples taken at baseline and at the end of the 24 h dwell time were analyzed for urea, creatinine, glucose, uric acid, magnesium, calcium, phosphorus, bicarbonate and albumin, globulin, total protein, Total bilirubin, alkaline phosphatase, and lactic dehydrogenase at the central laboratory of the IMSS Centro Medico Nacional (CMN) Siglo XXI Cardiology Hospital, Mexico City, Mexico. Blood samples taken during dwell time were analyzed immediately using a point-of-care analysis device (Instrumentation Laboratory Gem Premier 3500 Blood Gas Analyzer) for pH, pO2, PCO2, bicarbonate, sodium, potassium and hemoglobin. The treatment was to be considered safe if a subject did not experience any serious adverse events related to the infusate (defined according to Official Mexican Standard 012-SSA3-2012).

Tolerability was assessed by a patient questionnaire measuring pain on a numerical scale of 0-4 (at infusion start; at 1, 3, and 5 minutes from infusion start; at the end of infusion; 15 and 30 minutes after infusion end; every hour after infusion end thereafter, until end of dwell time and at the start of drain; 5 minutes later and at the end of the drain) and by application of the McGill pain questionnaire at 30 min after the end of the infusion. The treatment considered tolerable if a subject completed the 24-hour dwell time without reporting grade 3-4 pain that led to discontinuation of the dwell.

Efficacy was evaluated as the ultrafiltration (UF) volume and the total sodium excretion assessed by 1) total ultrafiltration volume at the end of the 24-hour dwell and 2) total sodium excretion at the end of the 24-hour dwell (concentration of sodium in IP fluid sample at end of dwell time multiplied by total drained volume). Sodium removal was monitored by measuring sodium concentration in peritoneal dialysis liquid at every 15 min to every hour during dwell time. Change of plasma glucose concentration was assessed by blood analysis. All patients provided informed consent prior to study procedures.

Primary peritoneal liquid samples labeled with patient identification and timepoint of collection were collected and analyzed offsite for icodextrin, potassium, bicarbonate, sodium, glucose and other analytes. Sodium, glucose, and icodextrin concentrations in IP fluid were determined using a Roche fully automated chemistry autoanalyzer (Roche Diagnostics, Indianapolis, USA). Icodextrin in PD fluid and serum was measured following hydrolysis of all glucose polymers to glucose using amyloglucosidase. Briefly, 100 μl of infusate was incubated with 500 μl of amyloglucosidase (6 mg/ml) at 55° C. for 30 minutes, the samples were diluted 20-fold for infusate and 5-fold for plasma. Glucose was measured using the Roche analyzer. I-131-albumin concentration in IP fluid was determined by gamma counting using either a Cobra Gamma Counter (Canberra-Packard Corp., Schwadorf, Austria) or a Daxor BVA device (Daxor Inc., New York, NY, USA). Peritoneal volumes were calculated based on icodextrin concentration, the known instilled volume, and total ultrafiltration volume. The absolute quantity of IP solute was calculated using the I-131 albumin derived volume multiplied by the solute concentration in the peritoneal fluid. Net UF was calculated by subtraction of volume instilled from total drained volume.

Upon completion of the 24-hour dwell time, the subjects' peritoneal cavity was drained completely. The patient's weight was measured before and after the drainage. Patients again were asked to empty their bladder and an echocardiogram and bio impedance was performed. Subsequently, patients resumed their regular PD prescriptions (outpatients: immediately; patients with automated PD but without recent infusion: immediately; patients with automatic PD and with recent infusion: until the next scheduled cycle on the same evening).

Continuous data is shown as mean±standard deviation (SD) or median (quartile 1-quartile 3) according to the observed distribution. Categorical data is shown as frequency (percentage). Statistical significance was defined as 2-tailed P<0.05. Statistical analyses were performed with IBM SPSS Statistics version 26 (IBM Corp, Armonk, NY) and Stata SE version 16.0 (StataCorp, College Station, TX).

Demography and baseline characteristics for the group of ten patients that completed the study are set forth in the following below. All patients completed the 24-hour dwell time with a 30% icodextrin/10% dextrose IP solution.

| Category | Subjects (n = 10) |
|---|---|
| Age (Year) | |
| Median (Interquartile Range-IQR) | 58 (18) |
| Sex, n (%) | |
| Male | 7 (70) |
| Female | 3 (30) |
| Medical History, n (%) | |
| Diabetes type 2 | 6 (60) |
| Hypertension | 10 (100) |
| Smoker | |
| active | 0 (0) |
| suspended | 4 (40) |
| Hyperlipidemia | 7 (70) |
| Abdominal surgery | 4 (40) |
| Heart Failure | 0 (0) |
| Kidney transplant | 0 (0) |
| Alcohol or illicit drug use | 0 (0) |
| End stage renal disease | |
| Etiology of kidney failure, n (%) | |
| Diabetes Type 2 | 6 (60) |
| Hypertension | 0 (0) |
| Glomerulonephritis | 0 (0) |
| Idiopathic | 3 (30) |
| Other (nephrolithiasis) | 1 (10) |
| Duration of PD requirement (years) | |
| Median (IQR) | 2 (3) |
| Type of PD, n (%) | |
| CAPD | 1 (10) |
| APD | 9 (90) |
| Number of PD bags/day, n (%) | |
| 1 | 0 (0) |
| 2 | 9 (90) |
| 3 | 0 (0) |
| >4 | 1 (10) |

Data presented as N (%) and median and interquartile range (IQR) as indicated. PD=peritoneal dialysis; CAPD=Continuous ambulatory peritoneal dialysis; APD=ambulatory peritoneal dialysis

All 10 patients completed the 24-hour dwell and the infusate was well tolerated with mild pain being reported mainly during infusion, thus fulfilling criteria that the infusate was tolerable. Median total drained volume was 3031.5 ml (IQR 528 mL), with a median total of 387 mmol (IQR 62 mmol) of sodium removed. Total excretion of chloride was lower than that of sodium, which is an advantage of this treatment as compared with loop diuretic therapy where chloride wasting is common further worsening diuretic resistance.

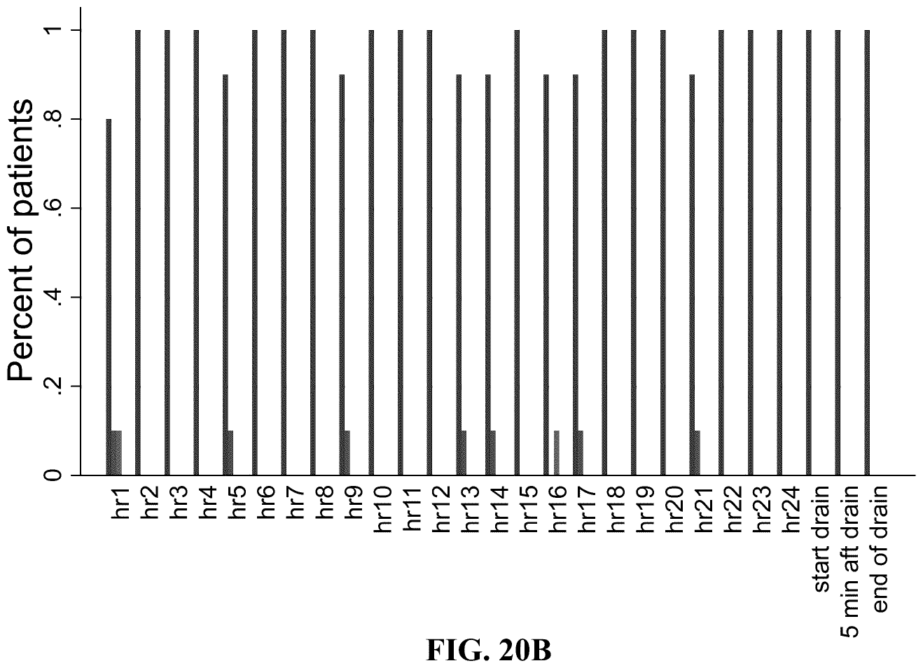

As shown in the table below, no patients died or experienced a serious adverse event. In total, 22 adverse events occurred, of which 18 (81.8%) were deemed at least possibly related to study treatment. Three patients with severe hypertension at baseline had a systolic blood pressure decrease of 40 mmHg or more, but no patients had a systolic blood pressure less than 100 mHg or symptoms of hypotension. Overall, the test solution was well tolerated by the participants with only 1 patient reporting significant pain (FIGS. 20A-20B), which occurred at the end of intraperitoneal instillation of the test solution and resolved within 15 minutes post infusion.

| | Patients |
|---|---|
| Adverse Events, N (%) | (N = 10) |
| Death | 0 (0%) |
| Serious Adverse Events | 0 (0%) |
| Non-serious Adverse Events | |
| Abdominal pain | 8 (80%) |
| Cramps | 3 (30%) |
| Nausea | 1 (10%) |
| Vomiting | 2 (20%) |
| Gastric fullness | 1 (10%) |
| Diaphoresis | 1 (10%) |
| Decrease in SBP > 40 mmHg | 3 (30%) |
| without Hypotension* | |
| SBP < 100 mmHg | 0 (0%) |
| Symptomatic hypotension | 0 (0%) |
| Anxiety | 1 (10%) |
| Pruritus | 1 (10%) |
| Chills | 1 (10%) |
| Discontinuation of Therapy | |
| due to Adverse Events | |
| Serious Adverse Event | 0 (0%) |
| Non-Serious Adverse Event | 0 (0%) |

Data presented as N (0%) during the 24-hour dwell time. SBP=systolic blood pressure. *In these 3 patients, the average SBP at baseline was 186 mmHg (>175 mmHg in all), and the SBP decreased to an average SBP of 1117 mmHg (≥110 mmHg in all). No patients had symptoms of hypotension.

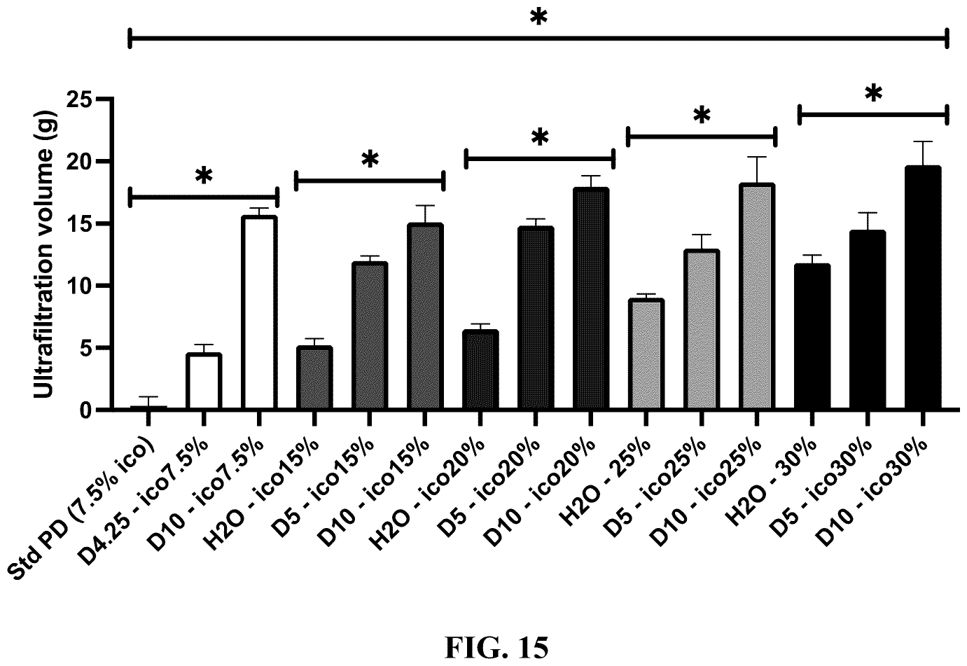

Median sodium concentration in the peritoneal dialysis liquid at the end of the 24-hour dwell time was 130 mmol/L (IQR 6; N=10). Median total amount of sodium removed as calculated by multiplication with the total volume drained from each subject was 387 mmol (IQR 62 mmol). FIGS. 8, 9 and 10 are graphs of sodium, icodextrin and glucose concentrations in the peritoneal fluid at the measurement time points during the study. In particular, FIG. 8 shows that the sodium concentration, rises from zero to about 110 mmol/L within 2 hours of instilling the infusate in the peritoneal cavity. FIG. 9 indicates that the concentration of the icodextrin in the peritoneal fluid declines rapidly in the first 3 hours, then continues slowly declining during the remainder of the dwell period. FIG. 10 also displays a rapid decline after initial instillation, and declines asymptotically towards zero.

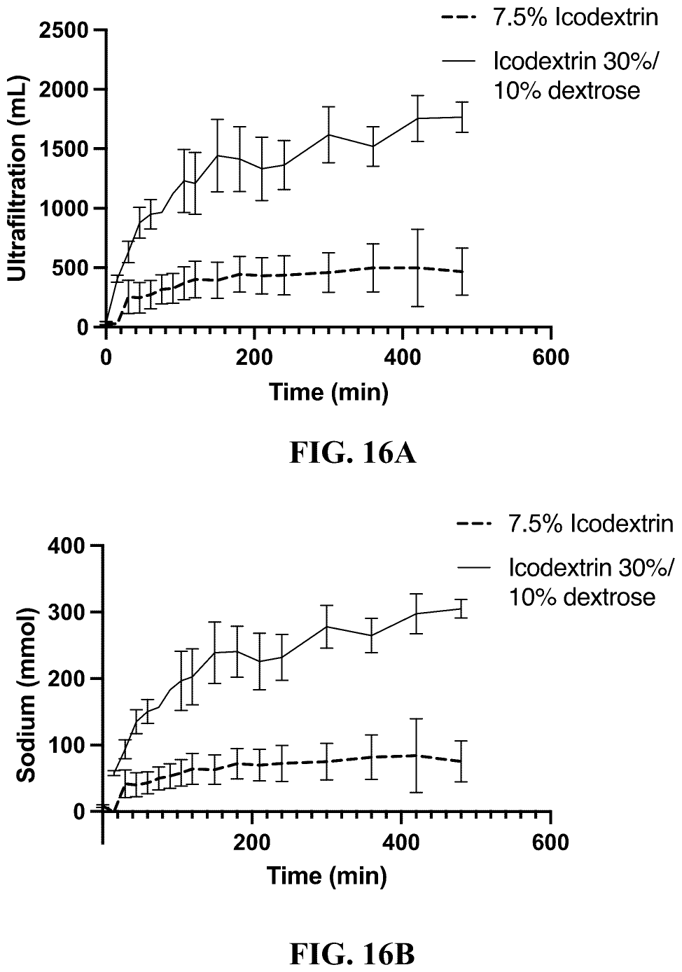

Referring now to FIGS. 11-14, the pharmacokinetics of the icodextrin as a function of time during the dwell period are discussed. Dots are mean values and error bars correspond to standard errors of the mean. In particular, FIG. 11 displays the icodextrin concentration in the patient's plasma; FIG. 12 describes the icodextrin concentration in peritoneal dialysis fluid; FIG. 13 displays the estimated percent of drained volume; and FIG. 14 corresponds to the estimated total amount of icodextrin present in the peritoneal cavity. Peritoneal volumes utilized for FIGS. 13 and 14 were computed by assuming a linear loss of fluid from the peritoneal fluid cavity to account for the measured absorption of icodextrin (total mass of icodextrin infused vs. total mass of icodextrin recovered in the drain). In all cases, the abscissa represents hours of dwell time.

FIGS. 11 and 12, taken together, demonstrate an unexpected advantage of using a high concentration of icodextrin in a relatively small (500 ml) of infusate that also includes dextrose. Specifically, the rapid decrease in icodextrin concentration in the peritoneal fluid, as shown in FIG. 12, compared to the relatively steady rise in icodextrin concentration in plasma results from the rapid influx of ultrafiltrate into the peritoneal cavity shortly after the DSR infusate is instilled. This is consistent with the rapid absorption of glucose (see FIG. 10) across the peritoneal membrane, while the bulk of the icodextrin remains in the peritoneal cavity but its concentration drops due to dilution caused by the influx of water.

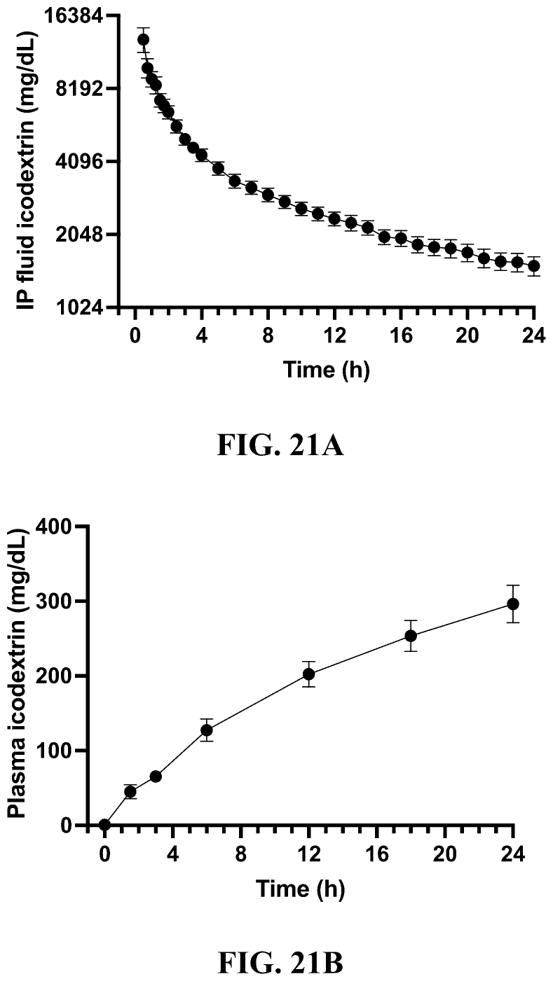

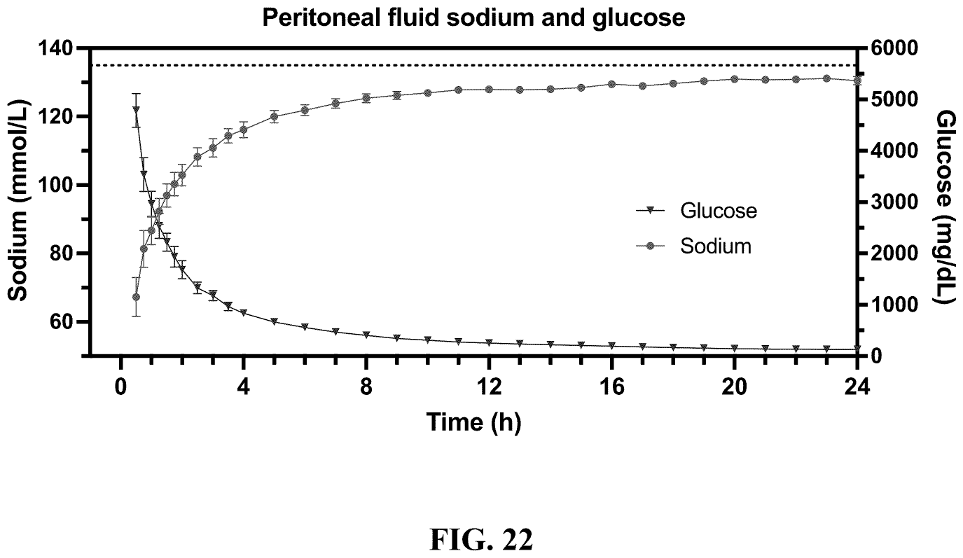

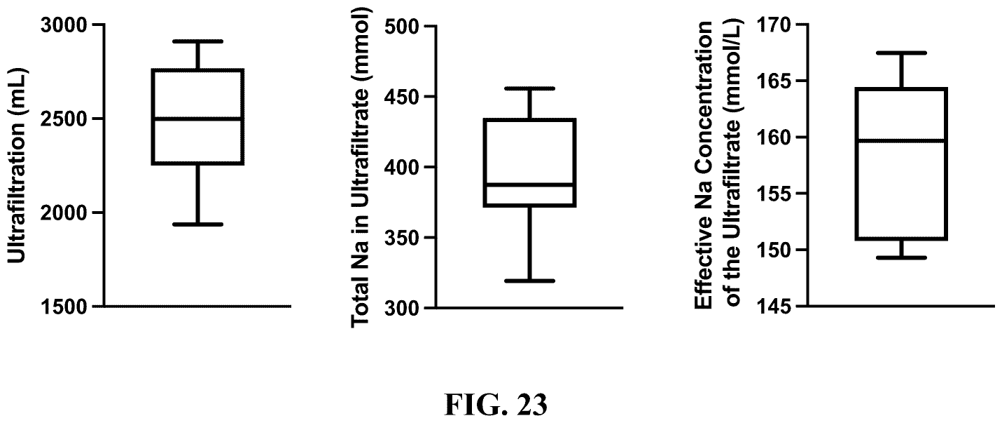

In addition, the icodextrin IP concentration decreased over the 24-hour study period as the UF volume increased (FIG. 21A-21B). Plasma levels of icodextrin and non-glucose icodextrin metabolites increased over the 24-hour dwell. The IP fluid sodium concentration increased rapidly over the about the first 8 hours with a slower rate of increase over the remainder of the 24-hour period (FIG. 22). Median total volume drained from the peritoneal cavity after a 24-hour dwell time was 3031.5 mL (2781-3253 mL), resulting in a median net UF of 2498 mL (Interquartile range of 2249-2768). The median serum sodium concentration was 131.0 mmol/L (127.5-133.7 mmol/L). The median total quantity of sodium removed at the end of the 24-hour dwell was 387 mmol (372-434 mmol) (FIG. 23). With 387 mmol of sodium removed and 2498 ml of water removed, the effective sodium concentration of the ultrafiltrate was significantly hypertonic to plasma (FIG. 23).

More specifically, it was observed that dextrose quickly is absorbed across the peritoneal membrane into the body primarily via diffusion. As a result, the clearance of dextrose is directly proportional to the concentration of glucose as such diffusion will increase proportionately to the concentration gradient. Because the peritoneal membrane is not permeable to icodextrin, clearance of icodextrin from the peritoneal cavity occurs primarily via pumping of peritoneal fluid at a continuous rate by the lymphatic system (e.g., 50-100 ml/hour of fluid). While clearance of large volumes of dilute icodextrin (e.g., 2L of 7.5% icodextrin) would be very slow, the use of small volumes of infusate having a high concentration of icodextrin would be disadvantageous. This would be so because the fluid removed by the lymphatics system shortly after instillation would have a very high concentration of icodextrin (e.g., 100 ml of 30% icodextrin contains much more icodextrin than 100 ml of 7.5% icodextrin).

To counteract this effect, it was observed that inclusion of a fast acting osmotic agent, such as dextrose, in the high concentration icodextrin infusate provides a number of important advantages, With the addition of 10 wt % dextrose, when 500 ml of the above-described infusate is instilled into the peritoneum, the dextrose component, due to its osmotic properties, quickly drags water and sodium into the abdominal cavity before it migrates through the peritoneal wall and is metabolized. This results in rapid dilution of icodextrin, by up to four times, such that the icodextrin remaining in the peritoneal cavity starts pulls water and sodium by its oncotic forces. Accordingly, 500 ml of 30 wt % icodextrin infusate expands rapidly to a larger volume of fluid, such that the rate at which icodextrin is removed by the lymphatic system is greatly reduced.

By comparison, use of a dilute icodextrin solution, e.g., 7.5 wt %, would require instillation of a much larger volume of fluid to prevent the rapid absorption issue noted above. Moreover, instillation of a large volume of low or no sodium infusate may promptly cause hyponatremia by drawing a large amount of sodium to the peritoneum, without a corresponding amount of free water. While providing a higher sodium concentration in a dilute icodextrin infusate would reduce the risk of hyponatremia, it also would reduce the efficacy of the DSR treatment with respect to sodium removal.

The fourth study was conducted with a no sodium infusate. However, it is theorized that a 30 wt % icodextrin/10 wt % dextrose infusate could be as much as isotonic for sodium (i.e., have sodium concentration of about 132 mmol/l) and still retain the majority of its efficacy in removing excess water. This is so because the solution produces such a powerful ultrafiltration effect (increasing volume about fivefold), of which only about 20% of the efficacy is driven by sodium diffusion vs. convective forces with ultrafiltration.

In conclusion, a 30% icodextrin/10% dextrose IP solution was safe and removed 387 mmol (˜9 grams) of sodium over 24 hours. Collectively, these results support future studies of a 30% icodextrin/10% dextrose IP solution as a non-renal sodium removal therapy for patients with edematous disorders such as HF.

Icodextrin is FDA approved for peritoneal administration at lower concentrations to prevent post-surgical adhesions (ADEPT, 4% solution) and as a PD solution (EXTRANEAL, 7.5% solution) (EXTRANEAL (icodextrin) Peritoneal dialysis solution [package label]. Deerfield, IL: Baxter Healthcare Corporation; 2020; ADEPT (icodextrin 4% solution) Adhesion reduction solution [package label]. Deerfield, IL: Baxter Healthcare Corporation; 2020). However, these solutions contain nearly isotonic sodium concentrations (132-133 mmol/L) (EXTRANEAL (icodextrin) Peritoneal dialysis solution [package label]. Deerfield, IL: Baxter Healthcare Corporation; 2020; ADEPT (icodextrin 4% solution) Adhesion reduction solution [package label]. Deerfield, IL: Baxter Healthcare Corporation; 2020). Given the established safety of a 7.5% icodextrin PD solution, a 30% icodextrin/10% dextrose solution was formulated. One concern of higher icodextrin/dextrose concentrations is the effect on peritoneal health. The IP glucose concentration in humans was only greater than standard PD solutions for a brief period, and the IP icodextrin concentrations were comparable to 7.5% icodextrin PD therapy for the majority of the 24-hour dwell (Moberly J B, Mujais S, Gehr T, et al. Pharmacokinetics of icodextrin in peritoneal dialysis patients. Kidney Int Suppl. October 2002; (81):523-33. doi:10.1046/j.1523-1755.62.s81.5.x). Chronic exposure to these higher concentrations was not associated with peritoneal abnormalities in animal models.

The 30% icodextrin/10% dextrose IP solution enhanced sodium and fluid removal. Dextrose produces UF via aquaporin-1 and small pores, while icodextrin generates fluid transport via small pores, minimizing back filtration and sodium sieving after dextrose is metabolized or systemically absorbed (Morelle J, Sow A, Fustin C A, et al. Mechanisms of Crystalloid versus Colloid Osmosis across the Peritoneal Membrane. Journal of the American Society of Nephrology: JASN. July 2018; 29(7):1875-1886. doi:10.1681/ASN.2017080828). Icodextrin increases net UF volume and sodium removal compared to dextrose peritoneal solutions, and combinations of icodextrin with dextrose synergistically increase sodium and fluid removal (Morelle J, Sow A, Fustin C A, et al. Mechanisms of Crystalloid versus Colloid Osmosis across the Peritoneal Membrane. Journal of the American Society of Nephrology: JASN. July 2018; 29(7):1875-1886. doi:10.1681/ASN.2017080828; Fischbach M, Schmitt C P, Shroff R, Zaloszyc A, Warady B A. Increasing sodium removal on peritoneal dialysis: applying dialysis mechanics to the peritoneal dialysis prescription. Kidney international. April 2016; 89(4):761-6. doi:10.1016/j.kint.2015.12.032; Finkelstein F, Healy H, Abu-Alfa A, et al. Superiority of icodextrin compared with 4.25% dextrose for peritoneal ultrafiltration. Journal of the American Society of Nephrology: JASN. February 2005; 16(2):546-54. doi:10.1681/ASN.2004090793). In previous experiments in humans, 1 liter of 10% dextrose, zero sodium peritoneal solution for 2 hours removed on average 4.5 g of sodium with a net UF volume of 700 mL, producing a hypertonic net ultrafiltrate sodium concentration of 280 mmol/L⁷. By removing substantially more sodium than water, zero sodium dextrose-based solutions rely on the kidneys to eliminate free water and can potentially cause hyponatremia with repeated administration. In contrast, the 30% icodextrin/10% dextrose solution removed approximately 9 g of sodium with a net UF volume of ˜2,500 mL, producing a mildly hypertonic net ultrafiltrate sodium concentration of approximately 160 mmol/L. Furthermore, the 10% dextrose IP solution produced a rapid UF, with most sodium removal occurring in the first 2 hours of a 6-hour dwell (Rao V S, Turner J M, Griffin M, et al. First-in-Human Experience With Peritoneal Direct Sodium Removal Using a Zero-Sodium Solution: A New Candidate Therapy for Volume Overload. Circulation. Mar. 31, 2020; 141(13):1043-1053). In agreement with previous studies (Morelle J, Sow A, Fustin C A, et al. Mechanisms of Crystalloid versus Colloid Osmosis across the Peritoneal Membrane. Journal of the American Society of Nephrology: JASN. July 2018; 29(7):1875-1886. doi:10.1681/ASN.2017080828), we found a 30% icodextrin/10% dextrose solution resulted in a more controlled and sustained UF rate over 24 hours, potentially allowing a single dwell with sustained efficacy for a longer duration and intermittent treatments. Finally, the enhanced efficiency of a 30% icodextrin/10% dextrose solution may facilitate smaller peritoneal infusion volumes. In this study, a 500 ml icodextrin/dextrose solution produced greater sodium and fluid removal than a 1,000 ml dextrose solution in our previous animal models (Rao V S, Turner J M, Griffin M, et al. First-in-Human Experience With Peritoneal Direct Sodium Removal Using a Zero-Sodium Solution: A New Candidate Therapy for Volume Overload. Circulation. Mar. 31, 2020; 141(13):1043-1053. doi:10.1161/CIRCULATIONAHA.119.043062).

The findings described in this example have application to the treatment of humans with edematous disorders such as HF. Excess sodium and water are the predominant cause of HF symptoms and hospitalization, and loop diuretics are the cornerstone therapy for sodium and water removal (Hollenberg S M, Warner Stevenson L, Ahmad T, et al. 2019 ACC Expert Consensus Decision Pathway on Risk Assessment, Management, and Clinical Trajectory of Patients Hospitalized With Heart Failure: A Report of the American College of Cardiology Solution Set Oversight Committee. J Am Coll Cardiol. Oct. 15, 2019; 74(15):1966-2011. doi:10.1016/j.jacc.2019.08.001; McDonagh T A, Metra M, Adamo M, et al. 2021 ESC Guidelines for the diagnosis and treatment of acute and chronic heart failure. European heart journal. Aug. 27, 2021; doi:10.1093/eurheartj/ehab368; Writing Committee M, Heidenreich P A, Bozkurt B, et al. 2022 AHA/ACC/HFSA Guideline for the Management of Heart Failure: A Report of the American College of Cardiology/American Heart Association Joint Committee on Clinical Practice Guidelines. J Am Coll Cardiol. Mar. 24, 2022; doi:10.1016/j.jacc.2021.12.012). Loop diuretic resistance is common, causing a highly variable diuretic-induced natriuresis (Cox Z L, Testani J M. Loop diuretic resistance complicating acute heart failure. Heart failure reviews. January 2020; 25(1):133-145. doi:10.1007/s10741-019-09851-9; Hodson D Z, Griffin M, Mahoney D, et al. Natriuretic Response Is Highly Variable and Associated With 6-Month Survival: Insights From the ROSE-AHF Trial. JACC Heart Fail. May 2019; 7(5):383-391. doi:10.1016/j.jchf2019.01.007; Testani J M, Brisco M A, Turner J M, et al. Loop diuretic efficiency: a metric of diuretic responsiveness with prognostic importance in acute decompensated heart failure. Circulation Heart failure. Mar. 1, 2014; 7(2):261-70. doi:10.1161/CIRCHEARTFAILURE.113.000895; Rao V S, Ivey-Miranda J B, Cox Z L, et al. Natriuretic Equation to Predict Loop Diuretic Response in Patients With Heart Failure. J Am Coll Cardiol. Feb. 16, 2021; 77(6):695-708. doi:10.1016/j.jacc.2020.12.022; Mullens W, Damman K, Harjola V P, et al. The use of diuretics in heart failure with congestion—a position statement from the Heart Failure Association of the European Society of Cardiology. European journal of heart failure. February 2019; 21(2):137-155. doi:10.1002/ejhf.1369). In the ROSE-AHF trial (Renal Optimization Strategies Evaluation in Acute Heart Failure), high dose loop diuretics produced a median 24-hour sodium output of 3.6 g (interquartile range 1.9-6.0 g) (Hodson D Z, Griffin M, Mahoney D, et al. Natriuretic Response Is Highly Variable and Associated With 6-Month Survival: Insights From the ROSE-AHF Trial. JACC Heart Fail. May 2019; 7(5):383-391. doi:10.1016/j.jchf2019.01.007; Chen H H, Anstrom K J, Givertz M M, et al. Low-dose dopamine or low-dose nesiritide in acute heart failure with renal dysfunction: the ROSE acute heart failure randomized trial. Multicenter Study Randomized Controlled Trial, Research Support, N.I.H., Extramural. JAMA: the journal of the American Medical Association. Dec. 18 2013; 310(23):2533-43. doi:10.1001/jama.2013.282190). By comparison, a 2-hour dwell of 10% dextrose peritoneal solution removed >4 g of sodium and a 24-hour dwell of 30% icodextrin/10% dextrose solution removed approximately 9 g of sodium (Rao V S, Turner J M, Griffin M, et al. First-in-Human Experience With Peritoneal Direct Sodium Removal Using a Zero-Sodium Solution: A New Candidate Therapy for Volume Overload. Circulation. Mar. 31 2020; 141(13):1043-1053. doi:10.1161/CIRCULATIONAHA.119.043062). Importantly, 29% of the ROSE-AHF population treated with diuretics had a positive sodium balance, which was associated with increased mortality (Hodson D Z, Griffin M, Mahoney D, et al. Natriuretic Response Is Highly Variable and Associated With 6-Month Survival: Insights From the ROSE-AHF Trial. JACC Heart Fail. May 2019; 7(5):383-391. doi:10.1016/j.jchf2019.01.007). Given the large quantity of sodium removed and absence of significant inter-subject variability, peritoneal sodium removal may have a substantial advantage over diuretics in patients with HF and diuretic resistance. Likewise, patients receiving traditional PD complicated by refractory hypervolemia may benefit from intermittent therapy.

In sum, while the long-term safety with chronic therapy, and the efficacy in patients without end-stage renal disease remain to be stablished, the zero-sodium, 30% icodextrin/10% dextrose intraperitoneal solution described herein provided significantly greater ultrafiltration and sodium removal than traditional dialysate solutions. The promising in-human safety and efficacy results warrant future investigation as a sodium removal therapy in patients with heart failure.

### Example 3: Animal Feasibility Studies

This example describes the evaluation of a sodium-free 30% icodextrin/10% dextrose intraperitoneal (IP) solution for enhanced sodium removal in animal models (mice, sheep and pigs).

### Methods

The primary aim of the experiments in rats were to evaluate varying concentrations of icodextrin/dextrose sodium-free IP solutions for sodium/water removal. Experiments in pigs and sheep aimed to describe fluid volume and sodium removal kinetics. Experiments in mice and sheep aimed to describe the effect of chronic exposure to icodextrin on peritoneal health. For studies involving chronic exposure in animals, the 30% icodextrin and 10% dextrose IP solution (DSR Infusate 2.0) was manufactured by Infomed Fluids S.r.l. (Bucharest, Romania) in compliance with international standards for Chemistry, Manufacturing, and Controls product development.

All animal studies were conducted with approval of the Yale University Institutional Animal Care and Use Committee and according to regulations outlined in the USDA Animal Welfare Act (9 CFR, Parts 1, 2 and 3) and the conditions specified in Guide for the Care and Use of Laboratory Animals, Eighth Edition (ILAR publication, 2011, National Academy Press).

### Experiment in Rats

Naïve Sprague Dawley rats (Rattus norvegicus) (N=114) were injected intraperitoneally with 10 ml of water-based test solution containing combinations of icodextrin (7.5%, 15%, 25%, 30%) and dextrose (no dextrose, 5% or 10%). Treatment groups and sizes were as follows: 17 receiving 7.5% icodextrin, 21 receiving 15% icodextrin, 16 receiving 20% icodextrin, 19 receiving 25% icodextrin, and 17 receiving 30% icodextrin. Each icodextrin group was divided in 3 subgroups with no, 5%, or 10% dextrose. The control group included 24 rats receiving 10% dextrose.

Dwell time was 5 hours based on preliminary experiments showing similar UF volume at 4 and 6 hours, implying peak at 5 hours. At the end of the 5-hour dwell, animals were euthanized and weighed. The abdomen was then opened, fluid drained completely by gravity/manual expression followed by swabbing of the cavity with dry gauze, and the animals were weighed again. The difference between these two weights represented the intraperitoneal volume (instilled fluid+UF). The instilled volume was assumed to be 10 g based on the 10 ml fluid injection. Rats with fluid-filled ceca or diarrhea, indicating intra-cecal injection, were excluded from the analysis (explaining the differing sizes of groups).

### Experiment in Sheep and Pigs

The primary outcome for the experiments in sheep and pigs was fluid volume and sodium removal kinetics. Six naïve Dorset sheep (Ovis aries) were anesthetized with a combination of intramuscular ketamine, xylazine, and tiletamine/zolazepam (Telazol), intubated, and maintained on inhaled isoflurane. Animals (n=3 in each group) were exposed to two different IP solutions: 30% icodextrin/10% dextrose or 7.5% standard icodextrin infusate (Extraneal, Baxter Healthcare LTD). Two standard peritoneal dialysis (PD) catheters were placed in the hepatic gutter and pelvis via a small laparotomy. A 500 mL IP solution infusate was instilled into the abdomen with 5-10 microcuries of I-131 albumin (Daxor Inc., New York, NY) added as an indicator for dilution to determine the kinetics of sodium and fluid removal over time. IP fluid was sampled (1 mL) in duplicate every 15 min until 2 hours, then every 30 min until 180 minutes, then hourly until 8 hours. The pigs were euthanized at the end of 8-hour dwell time, fluid drained via suction, and the total fluid volume was measured.

### Good Laboratory Practice Experiments on Chronic Intraperitoneal Health

To evaluate the effects of chronic exposure to 30% icodextrin and 10% dextrose IP solution on the peritoneum, kidneys, omentum, and peritoneal cavity, experiments in mice and sheep were conducted. Mice experiments were conducted at NAMSA (Minneapolis, MN), a facility compliant with U.S. FDA Good Laboratory practices (Regulations, 21 CFR Part 58). Mice (Breed C57BL/6) (N=42) were randomly divided into 2 treatment groups (N=21 per group with equal sex distribution) to receive 30% icodextrin and 10% dextrose IP solution or 4.25% Dextrose Dianeal Low Calcium (2.5 mEq/L) PD Solution (control group). Treatment groups were administered repeated daily doses of the IP solution (Subgroup 1: 2 mL; Subgroup 2: 1 mL, Subgroup 3: 0.5 mL) via IP catheter over 30 days. The animals were observed for signs of toxicity immediately after injection and daily throughout the study duration. Body weights were measured prior to the initial administration, weekly, and prior to termination. At the end of the 30-day survival period, blood was collected for hematology and serum chemistry and the animals were humanely euthanized. A limited necropsy was performed, including the examination of the kidneys, peritoneum, omentum, peritoneal cavity, and abdominal organs. Select tissues were collected for histopathological analysis. Overall interpretation was based on the incidence and severity of abnormalities including behavioral and clinical abnormalities, body weight changes, mortality, and gross observations during necropsy, clinical pathology, and histopathological evaluation as compared to control mice.

Sheep experiments were conducted at WuXi AppTec (Suzhou) in Jiangsu, China, a facility compliant with U.S. FDA Good Laboratory practices (Regulations, 21 CFR Part 58). Small-tailed Han sheep (Ovis aries) (N=18) with weights ranging 37.0-64.8 kg were randomly divided into 3 treatment groups (N=6 per group) to receive 30% icodextrin and 10% dextrose TP solution 10 mL/kg (Group 1), 20 mL/kg (Group 2), or 4.25% Dextrose Dianeal Low Calcium (2.5 mEq/L) PD Solution 40 mL/kg (Group 3 or “control group”). Sheep had two catheters placed in the peritoneal space for serial IP solution administration and an intravenous catheter placed for fluid or electrolyte repletion as needed. Groups 1 and 3 (control group) were treated daily for 45 days. Due to animal welfare concerns because of massive fluid removal and repletion, Group 2 was treated for 30 days. Every 24 hours, the peritoneal cavity was emptied of fluid prior to solution administration via PD catheters. Complete blood counts and serum chemistries were measured at baseline, mid-treatment period, and at time of necropsy. At the end of the treatment period, animals were euthanized, and necropsies were performed. Gross lesions were recorded, and tissue samples were collected from the kidney, peritoneum, and omentum. Tissue samples were embedded in paraffin blocks, sectioned at 4-6 μm thickness, deposited on slides, and stained with hematoxylin and eosin (H&E). Slides were evaluated by a veterinary pathologist for the presence of fibrosis, angiogenesis, or any findings abnormal for the respective type of host tissue.

### Assays and Calculations

Primary peritoneal liquid samples labelled with subject ID and timepoint of collection were collected and shipped to Yale University for analysis of electrolytes, icodextrin, and other analytes. Sodium, glucose, and icodextrin concentrations in IP fluid were determined using a Roche fully automated chemistry autoanalyzer (Roche Diagnostics, Indianapolis, USA). Icodextrin in PD fluid and serum was measured following hydrolysis of all glucose polymers to glucose using amyloglucosidase. Briefly, 100 μl of infusate was incubated with 500 μl of amyloglucosidase (6 mg/ml) at 55° C. for 30 minutes, the samples were diluted 20-fold for infusate and 5-fold for plasma. Glucose was measured using the Roche analyzer. I-131-albumin concentration in IP fluid was determined by gamma counting using either a Cobra Gamma Counter (Canberra-Packard Corp., Schwadorf, Austria) or a Daxor BVA device (Daxor Inc., New York, NY, USA). Peritoneal volumes were calculated based on icodextrin concentration, the known instilled volume, and total ultrafiltration volume. The absolute quantity of IP solute was calculated using the I-131 albumin derived volume multiplied by the solute concentration in the peritoneal fluid. Net UF was calculated by subtraction of volume instilled from total drained volume.

### Statistical Analysis

Continuous data is shown as mean±standard deviation (SD) or median (quartile 1-quartile 3) according to the observed distribution. Categorical data is shown as frequency (percentage). For experiments in rats, t-test was used to assess the effect of icodextrin on intraperitoneal fluid mass. Linear regression was used to estimate the effects of water, 5% dextrose, or 10% dextrose on intraperitoneal fluid mass, as well as the linear trend of icodextrin concentration (7.5%, 15%, 20%, 25%, and 30%) in each of these groups. For experiments in pigs and sheep, t-test was used to assess the effect of icodextrin on total UF and total sodium removed. For GLP experiments (mouse or sheep), t-test was used to compare continuous variables between groups. Statistical significance was defined as 2-tailed P<0.05. Statistical analyses were performed with IBM SPSS Statistics version 26 (IBM Corp, Armonk, NY) and Stata SE version 16.0 (StataCorp, College Station, TX).

### Results

### Dose Ranging Experiment in Rats

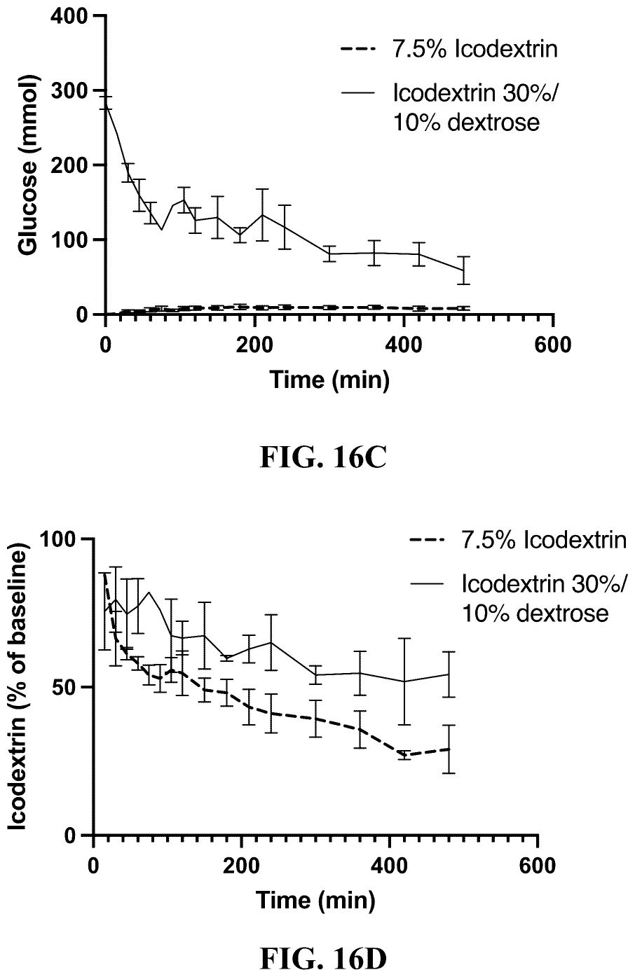

In rats, ascending concentrations of icodextrin and dextrose IP solutions resulted in progressively greater UF (p<0.001). (FIG. 15) The greatest UF volume was observed with a 30% icodextrin/10% dextrose IP solution. The combination of icodextrin and dextrose was additive with 30% icodextrin/10% dextrose producing 36% more UF than 30% icodextrin in water (p=0.006).

### Large Animal Evaluation

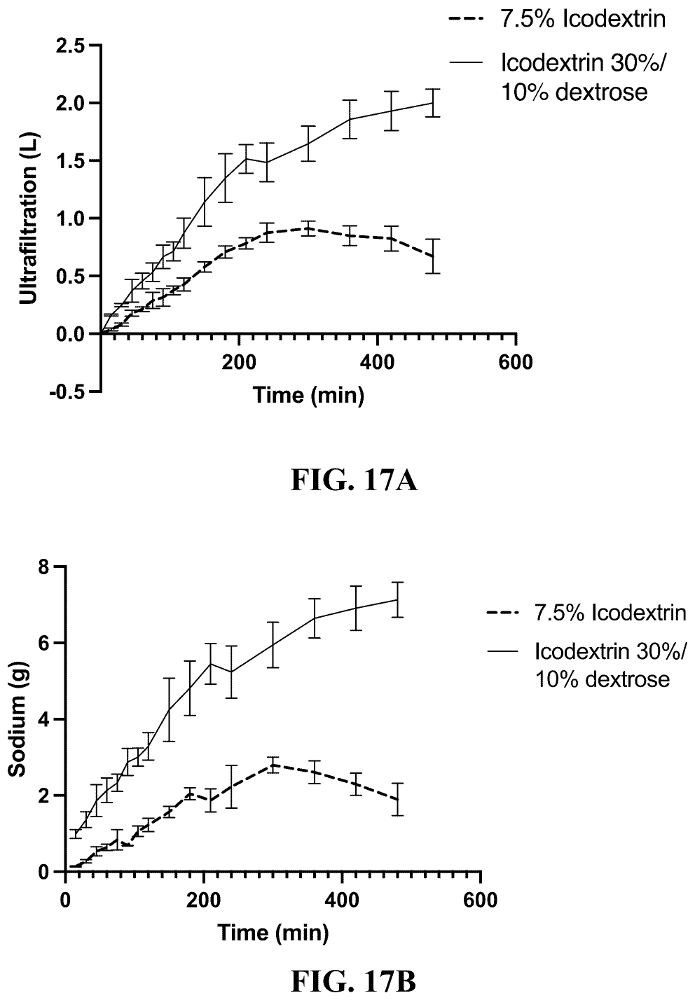

Total UF was more than 3.5 times greater in sheep infused with 30% icodextrin/10% dextrose compared with sheep infused with standard 7.5% icodextrin dialysate solution (Mean 1.77±0.22 L vs 0.47±0.34 L; p=0.005; FIGS. 16A-16D). Sheep that received 30% icodextrin/10% dextrose peritoneal solution compared to a standard 7.5% icodextrin peritoneal dialysis (PD) solution had greater ultrafiltration (FIG. 16A) and total sodium excretion (FIG. 16B). Glucose concentration was higher in the 30% icodextrin/10% dextrose group but decreased over time (FIG. 16C). Icodextrin concentration was also higher in animals that had received 30% icodextrin/10% dextrose than in those that had received standard 7.5% icodextrin PD solution (FIG. 16D), though concentration decreased in both groups over time. Total sodium removed was approximately 4 times greater with 30% icodextrin/10% dextrose compared with standard 7.5% icodextrin dialysis solution (7.07±0.72 g vs. 1.78±1.27 g; p=0.003).

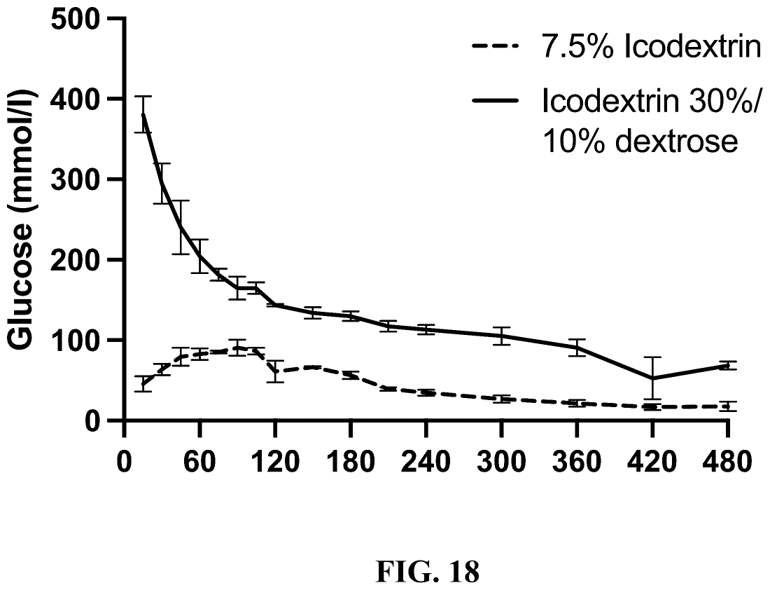

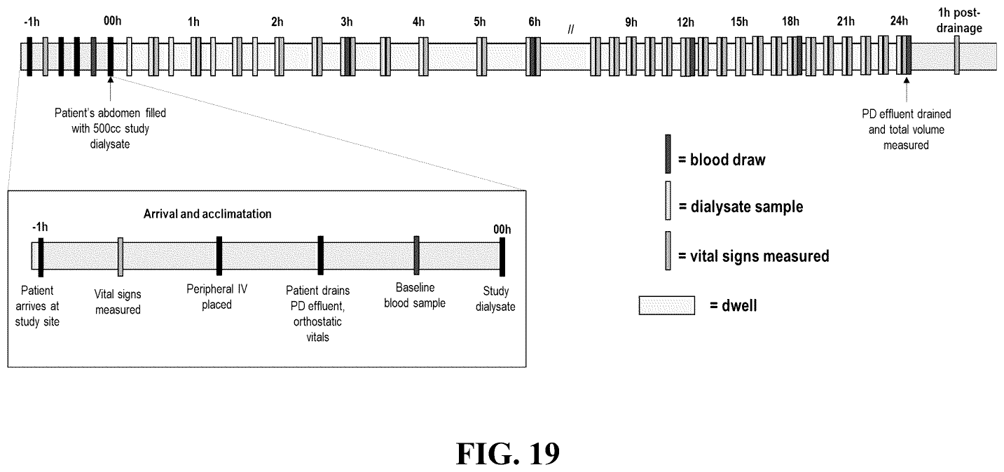

In pigs, ultrafiltration was higher with 30% icodextrin/10% dextrose in water peritoneal solution compared to 7.5% standard icodextrin PD solution (FIGS. 17A-17B). Total sodium excretion across the peritoneal membrane was also higher with 30% icodextrin/10% dextrose in water peritoneal solution (FIG. 17A). Glucose concentrations in peritoneal fluid were significantly increased in pigs that received 30% icodextrin/10% dextrose infusate compared to those that received a standard 7.5% icodextrin peritoneal dialysis (PD) solution (FIG. 18). There was significant intra-peritoneal icodextrin metabolism in the pig models but not in sheep (FIGS. 16-18), indicating that swine are not an ideal model to study icodextrin kinetics.

### Chronic Peritoneal Effects of Icodextrin

In mice and sheep chronically exposed to 30% icodextrin and 10% dextrose IP solution, no significant differences in the gross appearance or histopathology of peritoneum, kidney, omentum or peritoneal cavity were observed between icodextrin and control groups.

### CONCLUSION

The key findings from experiments with a novel zero-sodium icodextrin/dextrose IP solution in different animal models are: 1) ascending concentrations of icodextrin and dextrose significantly increased UF; 2) a 30% icodextrin/10% dextrose solution produced more than 3-fold greater sodium removal and UF volume compared to commercially available PD solutions; and 3) chronic exposure of animals to a 30% icodextrin/10% dextrose IP solution did not result in peritoneal tissue changes compared to control PD solutions. Collectively, these foundational results support future studies of a 30% icodextrin/10% dextrose IP solution as a non-renal sodium removal therapy for patients with edematous disorders such as HF.

While various illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. An infusate for direct sodium removal (DSR) therapy comprising: water, about 29% to about 31% icodextrin, about 9% to about 11% dextrose, and low to zero sodium.

2. The infusate of claim 1, wherein the infusate comprises about 9.5% to about 10.5% dextrose.

3. The infusate of claim 1 or claim 2, wherein the infusate comprises 10% dextrose.

4. The infusate of any one of claims 1-3, wherein the infusate comprises about 29.5% to about 30.5% icodextrin.

5. The infusate of any one of claims 1-4, wherein the infusate comprises 30% icodextrin.

6. The infusate of any one of claim 1-5, wherein the infusate has a sodium concentration lower than 135 meq/l.

7. An infusate for direct sodium removal (DSR) therapy comprising: water, 30% icodextrin, 10% dextrose, and low to zero sodium or a sodium concentration lower than 135 meq/l.

8. The infusate of any one of claims 1-7, further comprising a buffering agent.

9. A method of conducting direct sodium removal (DSR) therapy for a patient, the method comprising:
a. providing the infusate of any one of claim 1-8;
b. during a 14-28-day treatment period, on a first treatment day, instilling between 125 ml and 1.0 liter of the infusate into a peritoneal cavity of the patient;
c. retaining the infusate in the peritoneal cavity for a first 4-24-hour dwell period to induce sodium and ultrafiltrate to accumulate within the peritoneal cavity; and
d. removing the infusate, sodium and ultrafiltrate from the peritoneal cavity.

10. The method of claim 9, wherein the method further comprises
a. during subsequent treatment days, up to once daily following the first treatment day, instilling between 125 ml and 1.0 liter of the infusate into the peritoneal cavity of the patient;
b. retaining the infusate in the peritoneal cavity for subsequent 4-24-hour dwell periods during subsequent treatment days to induce sodium and ultrafiltrate to accumulate within the peritoneal cavity; and
c. removing the infusate, sodium and ultrafiltrate from the peritoneal cavity.

11. The method of claim 9 or 10, wherein the method further comprises, prior to the first treatment day, reducing or suspending administration of loop diuretics to the patient as compared to the patient's loop diuretic regimen prior to the treatment.

12. The method of any one of claims 9-11, wherein the method further comprises, following the 14-28-day treatment period removal, reducing or suspending administration of loop diuretics to the patient during a follow-up period of at least three months as compared to the patient's loop diuretic regimen prior to the treatment.

13. The method of any one of claims 9-12, wherein the infusate is instilled into the peritoneal cavity using a peritoneal dialysis catheter.

14. The method of claim 13, wherein removal of the infusate, sodium and ultrafiltrate from the peritoneal cavity following the first and subsequent 4-24 hour dwell period uses the peritoneal dialysis catheter.

15. The method of any one of claims 9-14, wherein removal of the infusate, sodium and ultrafiltrate from the peritoneal cavity following the first and subsequent dwell periods uses an implantable pump to transfer fluid from the peritoneal cavity to a bladder of the patient.

16. The method of any one of claims 9-15, wherein the infusate is instilled into the peritoneal cavity via a subcutaneous port.

17. The method of any one of claims 9-16, wherein removal of the infusate, sodium and ultrafiltrate from the peritoneal cavity following the first and/or subsequent 4-24-hour dwell periods uses an implantable pump to transfer fluid from the peritoneal cavity to a bladder of the patient.

18. The method of any one of claims 9-17, wherein the dextrose rapidly crosses a peritoneal membrane of the patient and draws water into the peritoneal cavity to dilute the concentration of icodextrin, thereby reducing a rate at which the icodextrin is removed from the peritoneal cavity.

19. The method of any one of claims 9-18, during the 14-28-day treatment period, on the first treatment day, instilling 500 milliliters of the infusate into the peritoneal cavity of the patient.

20. The method of any one of claims 10-19, wherein the method comprises, during subsequent treatment days, up to once daily following the first treatment day, instilling 500 milliliters of the infusate into the peritoneal cavity of the patient.

21. The method of any one of claims 9-20, wherein the method comprises retaining the infusate in the peritoneal cavity for the first 24-hour dwell period to induce sodium and ultrafiltrate to accumulate within the peritoneal cavity.

22. The method of any one of claims 10-21, wherein the method comprises retaining the infusate in the peritoneal cavity for subsequent 24-hour dwell periods during subsequent treatment days to induce sodium and ultrafiltrate to accumulate within the peritoneal cavity.

23. A method of conducting direct sodium removal (DSR) therapy for a patient, the method comprising:
a. providing the infusate of claim 7;
b. during a 14-28 day treatment period, on a first treatment day, instilling between 500 milliliters of the infusate into a peritoneal cavity of the patient;
c. retaining the infusate in the peritoneal cavity for a first 24-hour dwell period to induce sodium and ultrafiltrate to accumulate within the peritoneal cavity;
d. removing the infusate, sodium and ultrafiltrate from the peritoneal cavity;
e. during subsequent treatment days, up to once daily following the first treatment day, instilling between 500 milliliters of the infusate into the peritoneal cavity of the patient;
f. retaining the infusate in the peritoneal cavity for subsequent 24-hour dwell periods during subsequent treatment days to induce sodium and ultrafiltrate to accumulate within the peritoneal cavity; and
g. removing the infusate, sodium and ultrafiltrate from the peritoneal cavity.
